(19) United States
(12) Patent Application Publication
(10) Pub. No.: US 2013/0007916 A1
(43) Pub. Date: Jan. 3, 2013

(54) MODULATION OF LOW CARBON DIOXIDE INDUCIBLE PROTEINS (LCI) FOR INCREASED BIOMASS PRODUCTION AND PHOTOSYNTHESIS

(75) Inventor: Martin H. Spalding, Ames, IA (US)

(73) Assignee: IOWA STATE UNIVERSITY RESEARCH FOUNDATION, INC., Amse, IA (US)

(21) Appl. No.: 13/535,842

(22) Filed: Jun. 28, 2012

Related U.S. Application Data
(60) Provisional application No. 61/503,910, filed on Jul. 1, 2011.
(60) Provisional application No. 61/527,393, filed on Aug. 25, 2011.

(51) Int. Cl. C12N 15/79; A01H 5/00; C12N 5/10; C12N 15/82; C12N 15/74; C12N 1/13; A01H 1/06

(52) U.S. Cl. 800/290; 435/257.2; 800/298; 435/471; 435/468; 435/469; 435/470; 435/419

(57) ABSTRACT

The invention provides the disclosure of a novel plant/algae/cyanobacteria photosynthesis, biomass production, and productivity pathway involving low carbon dioxide inducible (LCI) proteins. According to the invention, the activity of one or more LCI proteins may be modulated to increase the same under conditions where such proteins are typically repressed. According to the invention, modulation of LCI protein activity was able to increase biomass production by as much as 80% under elevated CO₂ conditions. The invention includes methods, and genetically modified plants/algae/cyanobacteria, cells, plant parts and tissues.

### CROSS-REFERENCE TO RELATED APPLICATIONS

This application claims priority under 35 U.S.C. §119 to provisional applications Ser. No. 61/503,910 filed Jul. 1, 2011 and Ser. No. 61/527,393 filed Aug. 25, 2011, herein incorporated by reference in their entirety.

### GRANT REFERENCE

This invention was made with Government Support from the Department of Energy, DOE Grant No. DEAR0000010 and the Unites States Department of Agriculture, USDA Grant No. 20073531818433. The government has certain rights in the invention.

### FIELD OF THE INVENTION

The invention relates generally to the field of molecular biology.

### BACKGROUND OF THE INVENTION

Plant growth and development are controlled by intrinsic growth regulators or hormones and environmental cues through interconnected signal transduction pathways. A single hormone can regulate many different processes and likewise different hormones can cooperate to control the same cellular process. Aquatic photosynthetic organisms can modulate their photosynthesis to acclimate to CO₂-limiting stress by inducing a carbon-concentrating mechanism (CCM) that includes carbonic anhydrases and inorganic carbon (Ci) transporters. However, to date, Ci-specific transporters have not been well characterized in eukaryotic algae.

Accordingly, the ability to regulate photosynthesis, growth and plant biomass, is of considerable value to commercial agriculture. The present invention provides new mechanisms for stimulating plant growth, using the carbon dioxide transport pathway, to increase photosynthesis, and biomass production. These and other features will become apparent from the description of the invention which follows.

### SUMMARY OF THE INVENTION

Applicants have discovered that increasing the activity of CCM,-associated, low carbon inducible (LCI) proteins, particularly under conditions where the same are traditionally repressed, such as elevated carbon dioxide (CO₂) concentrations, can increase biomass production, photosynthesis and growth in plants/algae.

LCI proteins in Chlamydomonas include a variety of different protein types such as LCIB:low-Ci up-regulated soluble chloroplast protein, which is not a Ci transporter, but critical for internal Ci accumulation (see Wang & Spalding 2006 PNAS; Duanmu et al. 2009 Plant Physiol); LCIA (NAR1.2); low-Ci induced chloroplast envelope protein, closely related to nitrite/formate transporters, implicated in Ci transport by reported expression in Xenopus (Mariscal et al. 2006 Protist) and by RNAi knockdown (Duanmu et al. 2009 PNAS); HLA3(MRP1): low-Ci induced plasma membrane protein from the Mrp subfamily of ABC transporter superfamily, as demonstrated by RNAi knockdown to be directly or indirectly involved in bicarbonate transport (Duanmu et al. 2009 PNAS); LCI1: low-Ci induced plasma membrane protein, No obvious homologs even in other microalgae; possible structural analogs, Over-expression increases photosynthetic Ci assimilation (Ohnishi et al. 2010 Plant Cell); CCP1/CCP2: low-Ci induced chloroplast envelope proteins, Mitochondrial carrier protein superfamily, RNAi knockdown results in only minor phenotype (Pollock et al. 2004 Plant Molec. Biol); CAH1: periplasmic carbonic anhydrase (CA), implicated in provision of CO₂ or bicarbonate for plasma membrane Ci transporters; CAH3: thylakoid lumen CA required for dehydration of accumulated bicarbonate.

Applicants have demonstrated that overexpression of one or more LCI genes such as LCIA and/or LCIB increases biomass production, photosynthesis and growth in algae under elevated CO₂ conditions, where the expression of both genes normally is repressed. This has implications for increasing the productivity of algae and cyanobacteria, including those used for production of biofuel and other carbon based bio products, since most algae have LCI analogs, and even those without direct sequence analogs, including cyanobacteria, have functional analogs that function in their CCMs. This also has implications for increasing the productivity of plants, since they are expected to have a significant positive impact on photosynthesis as added transgenes, as LCI analogs are present in several plant species, Arabidopsis, for one example. In one aspect, the microalgae is Chlamydomonas reinhardtii, in another aspect, the microalgae is Ankistrodesmus, Botryococcus, Chlorella, Cyclotella, Dunaliella, Haematococcus, Nannochloropsis, Phaeodactylum, Porphyridium, Scenedesmus, Thalassiosira, or Volvox.

In other embodiments, the endogenous or heterologous LCI genes may be modulated to increase biomass in starch mutants, as the increase in biomass is largely starch, to increase instead desirable plant/algae products such as fatty acids.

It has been previously suggested that the LCIA protein may transport bicarbonate ions across the chloroplast envelope in algae in limiting CO₂ acclimated cells. The LCIB protein has been implicated in the scavenging of internal CO₂ in Chlamydomonas. Applicants postulated that overexpression of either LCIA or LCIB genes or a combination thereof under conditions where they are normally repressed would lead to increased photosynthesis, growth and productivity.

According to the invention, applicants have shown that constitutive overexpression of the LCIA and/or LCIB genes in algae resulted in increased biomass production by as much as 50% when grown in a photo bioreactor enriched with elevated CO₂ concentrations.

Applicants have identified a new method for improvement of plant/microalgae/cyanobacteria growth, photosynthesis, and biomass production by modulating activity of proteins involved in the inorganic carbon transport and accumulation pathway in conditions under which the activity of these proteins is typically repressed. As such methods are disclosed herein for improving plant/algae productivity and growth, photosynthesis, and biomass production by modulating the activity of the genes encoding one or more components of this pathway, particularly members of the family of low carbon inducible proteins, (LCI). Many members of this family have been identified and sequences are known to those of skill in the art and available through sources such as GENBANK and the like, such as LCIA (NAR1.2), LCIB, LCIC, LCIE, LCID, HLA3 (MRP1), LCI1, CCP1, and CCP2, CAH1, CAH3, etc. Examples include LCIA (formerly known as Nar 1.2) (Genbank AB168092, AF149737, AY612639), LCIB (Genbank AB168093, DQ49008), LCIC (Genbank AB168094, DQ7194), LCIE, (Gen bankDQ649007) LCID (DQ657198). Methods are also disclosed for identifying other components in this pathway which may be similarly modulated.

According to the invention, applicants have found that the activity of LCIA and/or LCIB may be modulated, in conditions under which the same are typically repressed or absent, in a plant/algae/cyanobacteria to improve plant/algae productivity and growth, photosynthesis, and biomass production when compared to a non-modulated plant/algae. Other family members such as LCIC, LCIE, LCID, HLA3 (MRP1), LCI1, CCP1, CCP2, CAH1, and CAH3, as well as variants and analogues and homologs from other plant/algae/cyanobacterial species will be expected to have similar affects.

The present invention therefore provides methods for enhancing photosynthesis and biomass production-related traits in plants/algae/cyanobacteria relative to control (non-modulated plants/algae/cyanobacteria), comprising preferentially modulating the activity of a CCM-related LCI protein, particularly LCIA and/or LCIB or a combination thereof or modulating the expression in a plant/algae/cyanobacteria or plant/algae/cyanobacteria part of a nucleic acid encoding one or more LCI protein particularly LCIA and/or LCIB or a combination thereof.

In other embodiments, different steps along this carbon transport pathway could be modulated such as any step which causes increase of CCM-related LCI activity or gene expression under certain external conditions or stages of development. For example, components found to affect this pathway to cause reduced activity under conditions of high carbon dioxide could be modulated, as could substrates, or signaling molecules associated with the same. The invention allows the identification of other signaling components that function in the pathway to regulate plant/algae/cyanobacteria photosynthesis, biomass production, and growth. These components can be identified as proteins, peptides or small molecules that interact with these LCI proteins by immunoprecipitation and/or yeast two-hybrid screens. These other signaling components can be also identified by screening for genetic modifiers (suppressors and enhancers) of mutants of these LCI genes.

In yet another embodiment, the method of modulating CCM-associated LCI activity including a LCI encoding polynucleotide which comprises, e.g., at least about 70%, at least about 75%, at least about 80%, at least about 85%, at least about 90%, at least about 95%, at least about 99%, at least about 99.5% or more sequence identity to sequences disclosed herein. Many plant/algae/cyanbacteria LCI proteins including LCIA, LCIB, LCIC, LCID, or LCIE from Chlamydomonas reinhardtii, are known to those of skill in the art and are readily available through sources such as GENBANK and the like, for nonlimiting examples include LCIA (formerly known as Nar 1.2) (Genbank AB168092, AF149737, AY612639), LCIB (Genbank AB168093, DQ49008), LCIC (Genbank AB168094, DQ7194), LCIE, (Gen bankDQ649007) LCID (DQ657198). Sequences and isolation and characterization of homologues by methods disclosed herein may also be obtained and used. Also included in the invention are CCM-related genes/proteins in algae and cyanobacteria which have a similar effect to LCIA and LCIB, even if not related to any of the LCI genes/proteins identified so far in Chlamydomonas. Such genes would be functionally analogous in that they would function to increase the concentration of internal CO₂ in the algae or cyanobacteria.

In another embodiment, the invention relates to methods for improving plant/algae/cyanobacteria productivity, biomass production and photosynthesis and the like by providing an isolated or recombinant modified plant/algae/cyanobacteria cell comprising at least one modification that modulates LCI activity including but not limited to one or more of LCIA, LCIB or a combination of the two.

In one embodiment, the methods involving a modification in the plant/algae/cyanobacteria cell include introducing at least one polynucleotide sequence comprising a LCI protein nucleic acid sequence, or subsequence thereof, into a plant/algae/cyanobacteria cell, such that the at least one polynucleotide sequence is operably linked to a promoter, and where the at least one polynucleotide sequence comprises, e.g., at least about 70%, at least about 75%, at least about 80%, at least about 85%, at least about 90%, at least about 95%, at least about 99%, about 99.5% or more sequence identity to a LCI sequence or a subsequence thereof, or a complement thereof.

In certain embodiments, a plant/algae/cyanobacteria cell resulting from the methods of the invention is from an algae, a cyanobacterium, a dicot or a monocot.

In yet another embodiment, the present invention is directed to a transgenic plant/algae/cyanobacteria or plant/algae/cyanobacteria cells with improved plant/algae/cyanobacteria productivity, biomass production and/or photosynthesis, containing the nucleic acids described herein. In one aspect, plant/algae/cyanobacteria is Chlamydomonas reinhardtii, in another aspect, the microalgae is Ankistrodesmus, Botryococcus, Chlorella, Cyclotella, Dunaliella, Haematococcus, Nannochloropsis, Phaeodactylum, Porphyridium, Scenedesmus, Thalassiosira, or Volvox. Preferred plants/algae/cyanobacteria grown from the methods of the present invention include but are not limited to maize, Arabidopsis, soybean, sunflower, sorghum, canola, wheat, alfalfa, cotton, oat, rice, barley, tomato, cacao and millet. Plants/algae/cyanobacteria produced according to the invention can have at least one of the following phenotypes as compared to a non-modified control plant/algae/cyanobacteria, at either normal or elevated carbon dioxide conditions, including but not limited to: increased dry weight, increased starch content, increased protein content, increased growth, as, for example measured by OD₇₅₀, increased plant height, increased root length, increased ear size, increased seed yield, increased seed size, or increased endosperm size when compared to a non-modified plant under similar conditions.

Detection of expression products is performed either qualitatively (by detecting presence or absence of one or more product of interest) or quantitatively (by monitoring the level of expression of one or more product of interest). Aspects of the invention optionally include monitoring an expression level or activity of a nucleic acid, polypeptide or chemical as noted herein for detection of the same in a plant/algae/cyanobacteria or in a population of plants/algae/cyanobacteria.

### BRIEF DESCRIPTION OF THE FIGURES

FIG. 1 depicts a transgenic Chlamydomonas lcib mutant line expressing an inserted LCIB gene regulated by a high expression, constitutive promoter that was crossed with a transgenic Chlamydomonas line expressing an inserted LCIA gene regulated by a high expression, constitutive promoter. Progeny from this cross with different genotypes, including the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background (LAB.wt-C4) and the LCIA and LCIB transgenes both in an lcib mutant background (LAB.pmp1-Aa1), were grown along with WT (21gr) in 200 ml photobioreactors to stationary phase, then harvested by centrifugation and the cell pellets dried for biomass determination. Values for each strain represent the average from two separate cultures in different mini-bioreactors, except that for 21gr, which represents the average of several separate cultures.

FIG. 2 shows additional data from experiment illustrated in FIG. 1. Starch accumulation in wild-type and transgenic Chlamydomonas strains grown to stationary phase in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background (LAB.wt-C4) and the LCIA and LCIB transgenes both in an lcib mutant background (LAB.pmp1-Aa1). Samples were removed just prior to the final harvest, centrifuged and the cell pellets assayed for starch content by determining glucose (Sigma GAHK-20 kit) after starch digestion with amyloglucosidase. Values for each strain represent the average from two separate cultures in different mini-bioreactors, except that for 21gr, which represents the average of several separate cultures.

FIG. 3 shows additional data from experiment illustrated in FIG. 1. Protein content of wild-type and transgenic Chlamydomonas strains grown to stationary phase in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background (LAB.wt-C4) and the LCIA and LCIB transgenes both in an lcib mutant background (LAB.pmp1-Aa1). Samples were removed just prior to the final harvest, centrifuged and the cell pellets assayed for total protein content using the Bio-Rad Protein Assay (Bio-Rad Laboratories, Hercules, Calif.). Values for each strain represent the average from two separate cultures in different mini-bioreactors, except that for 21gr, which represents the average of several separate cultures.

FIG. 4 shows additional data from experiment illustrated in FIG. 2. Cell density estimates for wild-type and transgenic Chlamydomonas strains determined over the course of culture growth in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background in two independent progeny (LAB.wt-C4 and LAB.Dc1) and the LCIA and LCIB transgenes both in an lcib mutant background (LAB.pmp1-Aa1). Samples were removed at frequent intervals, diluted into a measurable range and the OD₇₅₀ determined in a UV/Vis spectrophotometer. Values for each strain represent the average from two separate cultures in different mini-bioreactors, except that for 21gr, which represents the average of several separate cultures.

FIG. 5 shows a replication of experiment illustrated in FIG. 1. A transgenic Chlamydomonas lcib mutant line expressing an inserted LCIB gene regulated by a high expression, constitutive promoter was crossed with a transgenic Chlamydomonas line expressing an inserted LCIA gene regulated by a high expression, constitutive promoter. Progeny from this cross with different genotypes, including the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background in two independent progeny (LAB.wt-C4 and LAB.Dc1) and the LCIA and lcib transgenes both in an lcib mutant background (LAB.pmp1-Aa1), were grown along with WT (21gr) in 200 ml photobioreactors to stationary phase, then harvested by centrifugation and the cell pellets dried for biomass determination. Values for each strain represent the average from two separate cultures in different mini-bioreactors, except that for 21gr, which represents the average of several separate cultures.

FIG. 6 shows additional data from experiment illustrated in FIG. 5. Starch accumulation in wild-type and transgenic Chlamydomonas strains grown to stationary phase in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background in two independent progeny (LAB.wt-C4 and LAB.Dc1) and the LCIA and LCIB transgenes both in an lcib mutant background (LAB.pmp1-Aa1). Samples were removed just prior to the final harvest, centrifuged and the cell pellets assayed for starch content by determining glucose (Sigma GAHK-20 kit) after starch digestion with amyloglucosidase. Values for each strain represent the average from two separate cultures in different mini-bioreactors, except that for 21gr, which represents the average of several separate cultures.

FIG. 7 shows additional data from experiment illustrated in FIG. 5. Starch accumulation in wild-type and transgenic Chlamydomonas strains determined over the course of culture growth in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background in two independent progeny (LAB.wt-C4 and LAB.Dc1) and the LCIA and LCIB transgenes both in an lcib mutant background (LAB.pmp1-Aa1). Samples were removed at indicated intervals, centrifuged and the cell pellets assayed for starch content by determining glucose (Sigma GAHK-20 kit) after starch digestion with amyloglucosidase. Values for each strain represent the average from two separate cultures in different mini-bioreactors, except that for 21gr, which represents the average of several separate cultures.

FIG. 8 shows additional data from experiment illustrated in FIG. 5. Protein content of wild-type and transgenic Chlamydomonas strains grown to stationary phase in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background in two independent progeny (LAB.wt-C4 and LAB.Dc1) and the LCIA and LCIB transgenes both in an lcib mutant background (LAB.pmp1-Aa1). Samples were removed just prior to the final harvest, centrifuged and the cell pellets assayed for total protein content using the Bio-Rad Protein Assay (Bio-Rad Laboratories, Hercules, Calif.). Values for each strain represent the average from two separate cultures in different mini-bioreactors, except that for 21gr, which represents the average of several separate cultures.

FIG. 9 shows additional data from experiment illustrated in FIG. 5. Cell density estimates for wild-type and transgenic Chlamydomonas strains determined over the course of culture growth in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background in two independent progeny (LAB.wt-C4 and LAB.Dc1) and the LCIA and LCIB transgenes both in an lcib mutant background (LAB.pmp1-Aa1). Samples were removed at frequent intervals, diluted into a measurable range and the OD₇₅₀ determined in a UV/Vis spectrophotometer. Values for each strain represent the average from two separate cultures in different mini-bioreactors, except that for 21gr, which represents the average of several separate cultures.

FIG. 10 shows a transgenic Chlamydomonas lcib mutant line expressing an inserted LCIB gene regulated by a high expression, constitutive promoter that was crossed with a transgenic Chlamydomonas line expressing an inserted LCIA gene regulated by a high expression, constitutive promoter. Progeny from this cross with different genotypes, including the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background (LAB.wt-C4) and the LCIA and LCIB transgenes both in an lcib mutant background (LAB.pmp1-Aa1), were grown along with WT (21gr) in 200 ml photobioreactors to stationary phase, then harvested by centrifugation and the cell pellets dried for biomass determination.

FIG. 11 shows the over-expression of LCIA in 21 gr Strain (Northern blots) as LA-A, LA-B etc. 21 gr is the wild type strain, LciA is the low CO₂ inducible gene, putative Ci transporter, and HR promoter is the Hsp70-Rbsc2 promoter.

FIG. 12 shows Northern blot of LciA Gene in overexpression lines (LA22, LA23, etc.) and 21gr (gr).

FIG. 13 shows over-expression of LciB in pmp1 strain. LciB=a novel protein involved in inorganic carbon (Ci) accumulation pmp1-lcib mutant (wild type 137c background, 21gr-wildtype strain, HR promoter—Hsp70-Rbcs2 promoter.

FIG. 14 shows 21grLA-43×pmp1LB-16, PCR LciB: RbcSa-LciBas; LciA: LciAa-PsaDas.

FIG. 15 shows Progeny from cross (21grLA-43×pmp1LB-16) and their LCIB expression levels in western blots.

FIG. 16 shows Progeny from 21grLA-43×pmp1LB-16, PCR detection of LCIA and LCIB.

FIG. 17 shows overexpression of LCIB and LCIA, sfu/bamHi LciB into psp103, use Kpn1 to linearize.

FIG. 18 shows overexpression of LCIB in wild-type cw10.

FIG. 19 shows overexpression of LCIB in cw10-western blots of LCIB.

FIG. 20 shows overexpression of LCIB in cw10-western blots of LCIB.

FIG. 21 shows photosynthetic oxygen evolution.

FIG. 22 shows dry biomass (g/l±std. dev.) at stationary phase for WT (21gr) compared with double transgenic lines overexpressing both LCIA and LCIB (Ab4; Dc1; Dc2; C4; Aa1). Replicated (2-4 replicates for transgenics, 12 replicates for 21gr), photoautotrophic growth in standard medium under standard conditions. All double transgenics, except Ab4, are significantly different from 21gr at P<0.01.

FIG. 23 shows starch accumulation (g/l culture±std. dev.) at stationary phase for WT (21gr) compared with double transgenic lines overexpressing both LCIA and LCIB (Ab4; Dc1; Dc2; C4; Aa1). Replicated (2-4 replicates for transgenics, 12 replicates for 21gr), photoautotrophic growth in standard medium under standard conditions. All double transgenics are significantly different from 21gr at P<0.01.

FIG. 24 shows Photosynthetic rate measured as CO₂-dependent O₂ evolution (μmoles O₂ mg⁻¹ Chl h⁻¹) as a function of the dissolved inorganic carbon concentration (μM NaHCO₃, pH 7.0) for high-CO₂ acclimated WT (21gr; blue) compared with double transgenic line Dc2 (red) overexpressing both LCIA and LCIB. Four replicates from two independent experiments are shown.

FIG. 25 shows dry biomass (g DW/l±std. dev.) at stationary phase for WT (21gr) compared with double transgenic lines overexpressing both LCIA and LCIB (Aa1; C4), starch-deficient mutants, 21 sta1 and 21 sta6, and double transgenic lines overexpressing both LCIA and LCIB combined with the sta6 mutation (L4 and B3). Replicated (2-6 replicates for transgenics, >10 replicates for 21gr, 21sta1 and 21sta6), photoautotrophic growth in standard medium under standard conditions. All transgenic and mutant lines, except L4, are significantly different from 21gr at P<0.01.

FIG. 26 shows total fatty acid (FA) content (g FA/g DW±std. dev.) at stationary phase for WT (21gr) compared with double transgenic lines overexpressing both LCIA and LCIB (Aa1; C4), starch-deficient mutants, 21sta1 and 21sta6, and double transgenic lines overexpressing both LCIA and LCIB combined with the sta6 mutation (L4 and B3). Replicated (2-6 replicates for transgenics, >10 replicates for 21gr, 21sta1 and 21sta6), photoautotrophic growth in standard medium under standard conditions. All transgenic and mutant lines, except Aa1 and C4, are significantly different from 21gr at P<0.01.

FIG. 27 shows total fatty acid (FA) yield (g FA/1 culture) at stationary phase for WT (21gr) compared with double transgenic lines overexpressing both LCIA and LCIB (Aa1; C4), starch-deficient mutants, 21sta1 and 21sta6, and double transgenic lines overexpressing both LCIA and lcib combined with the sta6 mutation (L4 and B3). Replicated (2-6 replicates for transgenics, >10 replicates for 21gr, 21sta1 and 21sta6), photoautotrophic growth in standard medium under standard conditions.

FIG. 28 is a Schematic Model of Chlamydomonas CCM. LCIA is a putative chloroplast envelope bicarbonate transporter and LCIB appears to be required for trapping CO₂ into the stromal bicarbonate pool.

FIG. 29 (Expression of LCIA and LCIB in Transgenics) is a Western blot analysis of LCIA and LCIB expression in wild type (WT) 21gr and over-expression strains LA43 (over-expressing LCIA), LB16 (over-expressing LCIB), and Dc2 (over-expressing LCIA and LCIB).

FIG. 30 (Biomass and Starch Yield in Transgenics) is a graph showing that transgenes LCIA and LCIB increase biomass yield.

FIG. 31 is a graph showing extra biomass of transgenics accumulates as starch.

FIG. 32 is a graph showing that increased starch accumulates throughout growth of the culture, without nitrogen starvation. A. Biomass yield of photoautotrophic cultures at stationary phase for wild-type (WT) strains, 21gr and 2137, single gene transgenics, LA43 (LCIA), and LB16 (LCIB), and double transgenics (LCIA+LCIB), Aa1, Ab4, Cc4, Dc1 and Dc2. B. Starch content of photoautotrophic cultures at stationary phase for WT, 21gr and 2137, single gene transgenics, LA43 (LCIA), and LB16 (LCIB), and double transgenics (LCIA+LCIB), Aa1, Ab4, Cc4, Dc1 and Dc2. C. Starch content during photoautotrophic growth of cultures for WT 21gr and transgenics LA43 (LCIA), LB16 (LCIB) and Dc2 (LCIA+LCIB).

FIG. 33 is a graph showing Increased CO₂ Assimilation in Transgenics. Whole-bioreactor CO₂ assimilation increases in Transgenics.

FIG. 34 is a graph showing CO₂ assimilation rate per mg protein is increased in transgenics.

FIG. 35 is a graph showing total CO₂ assimilated is increased in transgenics. A. measurement of net, direct, in situ uptake of CO₂ by WT 21gr and transgenics LA43 (LCIA), LB16 (LCIB) and Dc2 (LCIA+LCIB) in photobioreactors. B. Calculated rate of net in situ CO₂ assimilation per mg of protein for WT 21gr and transgenic lines LA43 (LCIA), LB16 (LCIB) and Dc2 (LCIA+LCIB). C. Total in situ net CO₂ assimilation for WT 21gr and transgenics LA43 (LCIA), LB16 (LCIB) and Dc2 (LCIA+LCIB) over the full growth of cultures in photobioreactors.

FIG. 36 is a graph showing that adding a Starch-less Mutation Channels the Extra Carbon into Fatty Acids. Fatty acid (FA) content increases in starch synthesis mutants st1 and st6, as well as in double transgenic Aa1 crossed with st6.

FIG. 37 is a graph showing that biomass decreases because synthesis of 1.5 g of oil requires the same CO₂ assimilation as 5 g of starch.

FIG. 38 is a graph showing that is Increased FA accumulates throughout growth of the culture, without nitrogen starvation.

### DETAILED DESCRIPTION OF THE INVENTION

### Definitions

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges recited within the specification are inclusive of the numbers defining the range and include each integer within the defined range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5th edition, 1993). The terms defined below are more fully defined by reference to the specification as a whole.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

As used herein, “antisense orientation” includes reference to a duplex polynucleotide sequence that is operably linked to a promoter in an orientation where the antisense strand is transcribed. The antisense strand is sufficiently complementary to an endogenous transcription product such that translation of the endogenous transcription product is often inhibited.

The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid.

One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made.

Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). See also, Creighton (1984) Proteins W. H. Freeman and Company.

By “encoding” or “encoded”, with respect to a specified nucleic acid, is meant comprising the information for translation into the specified protein. A nucleic acid encoding a protein may comprise intervening sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant/algae, animal, and fungal mitochondria, the bacterium Mycoplasma capricolum, or the ciliate Macronucleus, may be used when the nucleic acid is expressed therein. When the nucleic acid is prepared or altered synthetically, advantage can be taken of known codon preferences of the intended host where the nucleic acid is to be expressed.

“Elevated CO₂ or High CO₂” conditions as used herein includes any system where the CO₂ concentration of air is greater than ambient, 360 mL/L (350-400 ppm). High CO₂ can be, but is not limited to 5000 mL/L or 5% in air vol/vol. (elevated).

As used herein “full-length sequence” in reference to a specified polynucleotide or its encoded protein means having the entire amino acid sequence of, a native (nonsynthetic), endogenous, biologically active form of the specified protein. Methods to determine whether a sequence is full-length are well known in the art including such exemplary techniques as northern or western blots, primer extension, S1 protection, and ribonuclease protection. See, e.g., Plant Molecular Biology: A Laboratory Manual, Clark, Ed., Springer-Verlag, Berlin (1997). Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

By “host cell” is meant a cell which contains a vector and supports the replication and/or expression of the vector. Host cells may be prokaryotic cells such as E. coli, or eukaryotic cells such as yeast, insect, amphibian, or mammalian cells.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “isolated” refers to material, such as a nucleic acid or a protein, which is: (1) substantially or essentially free from components that normally accompany or interact with it as found in its naturally occurring environment. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by nonnaturally occurring means to a locus of the genome not native to that nucleic acid. Nucleic acids which are “isolated” as defined herein, are also referred to as “heterologous” nucleic acids.

Unless otherwise stated, the term “LCI, LCIA, or lcib nucleic acid” means a nucleic acid comprising a polynucleotide (an “LCI, LCIA, or lcib polynucleotide”) encoding an LCI, LCIA, or lcib polypeptide with LCI, LCIA, or lcib activity and includes all conservatively modified variants, homologs, paralogs and the like. An “LCI, LCIA, or lcib gene” is a gene of the present invention and refers to a heterologous genomic form of a full-length LCI, LCIA, or lcib polynucleotide.

As used herein, “localized within the chromosomal region defined by and including” with respect to particular markers includes reference to a contiguous length of a chromosome delimited by and including the stated markers.

As used herein, “marker” includes reference to a locus on a chromosome that serves to identify a unique position on the chromosome. A “polymorphic marker” includes reference to a marker which appears in multiple forms (alleles) such that different forms of the marker, when they are present in a homologous pair, allow transmission of each of the chromosomes of that pair to be followed. A genotype may be defined by use of one or a plurality of markers.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules which comprise and substantially represent the entire transcribed fraction of a genome of a specified organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning—A Laboratory Manual, 2nd ed., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” includes reference to whole plants/algae, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen, and microspores. The class of plants/algae which can be used in the methods of the invention is generally as broad as the class of higher plants/algae amenable to transformation techniques, including both monocotyledonous and dicotyledonous plants/algae. Also included within the term plant is algae, including Archaeplastida such as Chlorophta (green algae), Rhodophyta (Red algae), Glaucophyta, Rhizaria, Excavata such as Chlorarachniophytes such as Euglenids, Chromista, Alveolata such as the Heterokonts, Bacillariophyceae (Diatoms), cyanobacteria, Axodine, Bolidomonas, Eustigmatophyceae, Phaeophyceae (Brown algae), Chrysophyceae (Golden algae), Raphidophyceae, Synurophyceae, Xanthophyceae (Yellow-green algae) Cryptophyta, Dinoflagellates, and Haptophyta. Particularly preferred is the green algae Clamydomonas.

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid (s) as the naturally occurring nucleotide (s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art.

The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not always entirely linear. For instance, polypeptides may be branched as a result of ubiquitization, and they may be circular, with or without branching, generally as a result of post translation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. A “plant/algae promoter” is a promoter capable of initiating transcription in plant/algae cells whether or not its origin is a plant/algae cell. Exemplary plant/algae promoters include, but are not limited to, those that are obtained from plants/algae, plant viruses, and bacteria which comprise genes expressed in plant/algae cells such Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may affect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions.

The term “LCI” in relation to a polypeptide refers to any of the CCM-associated, low carbon dioxide inducible proteins described or identified in the art as part of the carbon dioxide photosynthesis stress response pathway and can include family members which are designated LCIN wherein the N can be either a letter or a number including but not limited to LCIA, LCIB, LCIC, LCIE, LCID, HLA3 (MRP1), LCI1, CCP1, and CCP2, LCI1-LCI 30, including LCI2, LCI3, LCI16, and the like. Some of these proteins are also referred to as NAR1.2, CAH1 and CAH3.

The term “LCI, LCIA, or LCIB polypeptide” is a polypeptide which has LCI, LCIA, or LCIB, activity and refers to one or more amino acid sequences, in glycosylated or non-glycosylated form. The term is also inclusive of fragments, variants, homologs, alleles or precursors (e.g., preproproteins or proproteins) thereof which retain LCI activity. An “LCI, LCIA, or LCIB protein” comprises a LCI, LCIA, or LCIB polypeptide. “LCI, LCIA, lcib activity” means that the polypeptide is capable of modulating photosynthesis to acclimate to CO₂-limiting stress by inducing a carbon-concentrating mechanism (CCM) that includes carbonic anhydrases and inorganic carbon (Ci) transporters.

LCIA, also known as Nar1.2, is unrelated to the LCIB gene family although it has a similar name and also is induced in Chlamydomonas in low carbon dioxide conditions. LCIA is also part of a gene family in Chlamydomonas, the Nar1 gene family, which includes several genes putatively involved in nitrite transport (related to prokaryotic formate/nitrite transporters)—Nar1.2 is the only member so far implicated in bicarbonate transport (and thus in the carbon dioxide concentrating mechanism).

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “residue” or “amino acid residue” or “amino acid” is used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes reference to hybridization, under stringent hybridization conditions, of a nucleic acid sequence to as other biologics. Thus, under designated immunoassay conditions, the specified antibodies bind to an analyte having the recognized epitope to a substantially greater degree (e.g., at least 2-fold over background) than to substantially all analytes lacking the epitope which are present in the sample. Specific binding to an antibody under such conditions may require an antibody that is selected for its specificity for a particular protein. For example, antibodies raised to the polypeptides of the present invention can be selected from to obtain antibodies specifically reactive with polypeptides of the present invention. The proteins used as immunogens can be in native conformation or denatured so as to provide a linear epitope.

The term “stringent conditions” or “stringent hybridization conditions” includes reference to conditions under which a probe will hybridize to its target sequence, to a detectably greater degree than to other sequences (e.g., at least 2-fold over background).

Stringent conditions are sequence-dependent and will be different in different circumstances. By controlling the stringency of the hybridization and/or washing conditions, target sequences can be identified which are 100% complementary to the probe (homologous probing).

Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 3° C. for short probes (e.g., 10 to 50 nucleotides) and at least about 6° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 MNaCI, 1% SDS (sodium dodecyl sulphate) at 37 C, and a wash in 1× to 2×SSC (20×SSC=3.0 MNaCI/0.3 M trisodium citrate) at 50 to 55 C. Exemplary moderate stringency conditions include hybridization in 40 to 45% formamide, 1 MNaCI, 1% SDS at 37 C, and a wash in <RTI 0.5× to 1×SSC at 55 to 60 C. Exemplary high stringency conditions include hybridization in 50% formamide, 1 MNaCI, 1% SDS at 37 C, and a wash in 0.1×SSC at 60 to 65 C. Specificity is typically the function of post-hybridization washes, the critical factors being the ionic strength and temperature of the final wash solution. For DNA/DNA hybrids, the Tm can be approximated from the equation of Meinkoth and Wahl, Anal. Biochem., 138: 267-284 (1984): Tm=81.5 C+16.6 (log M)+0.41(% GC)−0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1 C for each 1% of mismatching; thus, Tm, hybridization and/or wash conditions can be adjusted to hybridize to sequences of the desired identity. For example, if sequences with >90% identity are sought, the Tm can be decreased 10 C. Generally, stringent conditions are selected to be about 5 C lower than the thermal melting point (Tm) for the specific sequence and its complement at a defined ionic strength and pH. However, severely stringent conditions can utilize a hybridization and/or wash at 1, 2, 3, or 4 C lower than the thermal melting point (Tm); moderately stringent conditions can utilize a hybridization and/or wash at 6, 7, 8, 9, or 10 C lower than the thermal melting point (Tm); low stringency conditions can utilize a hybridization and/or wash at 11, 12, 13, 14, 15, or 20 C lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45 C (aqueous solution) or 32 C (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel, et al., Eds., Greene Publishing and Wiley-Interscience, New York (1995).

As used herein, “transgenic plant/algae” includes reference to a plant/algae which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant/algae part or plant/algae, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant/algae breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between a polynucleotide/polypeptide of the present invention with a reference polynucleotide/polypeptide: (a)“reference sequence”, (b)“comparison window”, (c) “sequence identity”, and (d)“percentage of sequence identity”.

(a) As used herein, “reference sequence” is a defined sequence used as a basis for sequence comparison with a polynucleotide/polypeptide of the present invention. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide/polypeptide sequence, wherein the polynucleotide/polypeptide sequence may be compared to a reference sequence and wherein the portion of the polynucleotide/polypeptide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides/amino acids residues in length, and optionally can be 30, 40, 50, 100, or longer. Those of skill in the art understand that to avoid a high similarity to a reference sequence due to inclusion of gaps in the polynucleotide/polypeptide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the . . . in the GCG Wisconsin Genetics Software Package, Version 10 (available from Accelrys Inc., 9685 Scranton Road, San Diego, Calif., USA). The CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994).

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol., 215: 403-410 (1990); and, Altschul et al., Nucleic Acids Res. 25: 3389-3402 (1997).

Software for performing BLAST analyses is publicly available, e.g., through the National Center for Biotechnology Information www at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score.

Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5877 (1993)). One measure of similarity provided by the BLAST algorithm is the smallest sum probability (P(N)), which provides an indication of the probability by which a match between two nucleotide or amino acid sequences would occur by chance. BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17: 149-163 (1993)) and XNU (Claverie and States, Comput. Chem., 17: 191-201 (1993)) low-complexity filters can be employed alone or in combination.

Unless otherwise stated, nucleotide and protein identity/similarity values provided herein are calculated using GAP (GCG Version 10) under default values. GAP (Global Alignment Program) can also be used to compare a polynucleotide or polypeptide of the present invention with a reference sequence. GAP uses the algorithm of Needleman and Wunsch (J. Mol. Biol. 48: 443-453, 1970) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 100. Thus, for example, the gap creation and gap extension penalties can each independently be: 3, 4, 5, 6, 7, 8, 9, 10, 15, 20, 30, 40, 50, 60 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff & Henikoff (1989) Proc. Natl. Acad. Sci. USA 89: 10915).

Multiple alignment of the sequences can be performed using the CLUSTAL method of alignment (Higgins and Sharp (1989) CABIOS. 5: 151-153) with the default parameters (GAPPENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e. <RTI g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

### Regulation of the Carbon Transport Pathway

The invention comprises the discovery of a mechanism for regulation of the carbon transport system pathway for plant/algae/cyanobacteria photosynthesis and biomass production in plants/algae/cyanobacteria. As such methods are disclosed for improving plant/algae/cyanobacteria photosynthesis, biomass production, and productivity by modulating the activity of one or more components of this pathway, including members of the family of LCI proteins which have been identified herein. Methods are also disclosed for identifying other components in this pathway.

According to the invention, applicants have found that the LCI proteins, particularly LCIA and/or LCIB which are typically repressed in higher than ambient air carbon dioxide conditions, may be modulated to increase plant/algae/cyanobacteria photosynthesis, biomass production, and productivity when compared to a non-modulated plant/algae/cyanobacteria. Other family members, as well as analogues and homologues from other LCI proteins and other plant/algae/cyanobacteria species will be expected to have similar affects.

Thus, the invention in one aspect provides a method for enhancing yield-related traits such as plant/algae photosynthesis, biomass production, and productivity relative to control plants/algae, comprising modulating the activity or expression in a plant/algae/cyanobacteria of a nucleic acid encoding a LCI protein, or a part thereof.

The present invention therefore provides methods for enhancing yield-related traits in plants/algae/cyanobacteria relative to control plants/algae/cyanobacteria, comprising preferentially modulating the activity of a LCI protein, such as LCIA and/or LCIB or a combination thereof or modulating the expression in a plant/algae/cyanobacteria of a nucleic acid encoding one or more LCI proteins, such as LCIA and/or LCIB or a combination thereof.

In other embodiments, other steps along the plant/algae/cyanobacteria carbon transport pathway could be modulated. The invention allows the identification of other signaling components that function in the LCI pathway to regulate plant/algae/cyanobacteria photosynthesis, biomass production, and productivity and other processes. These components can be identified as proteins, peptides or small molecules that interact with these proteins by immunoprecipitation and/or yeast two-hybrid screens. These other signaling components can be also identified by screening for genetic modifiers (suppressors and enhancers) of mutants of these LCI proteins or components.

In another embodiment, the method of modulating LCI protein activity including LCIA and/or LCIB includes an LCIA and/or LCIB encoding polynucleotide which comprises, e.g., at least about 70%, at least about 75%, at least about 80%, at least about 85%, at least about 90%, at least about 95%, at least about 99%, at least about 99.5% or more sequence identity to sequences disclosed herein. Many plant/algae/cyanobacteria LCI proteins including LCIA and/or LCIB are known to those of skill in the art and are readily available through sources such as GENBANK, and by isolation and characterization of homologues by methods disclosed herein.

In another embodiment, the invention relates to methods for improving plant/algae/cyanobacteria yield traits such as photosynthesis, biomass production, productivity, and the like by providing an isolated or recombinant modified plant/algae/cyanobacteria cell comprising at least one modification that modulates LCI protein activity including LCIA and/or LCIB protein.

In one embodiment, the methods involving a modification in the plant/algae/cyanobacteria cell include introducing at least one polynucleotide sequence comprising a LCI protein such as LCIA and/or LCIB nucleic acid sequence, or subsequence thereof, into a plant/algae/cyanobacteria cell, such that the at least one polynucleotide sequence is operably linked to a promoter, and where the at least one polynucleotide sequence comprises, e.g., at least about 70%, at least about 75%, at least about 80%, at least about 85%, at least about 90%, at least about 95%, at least about 99%, about 99.5% or more sequence identity to sequences disclosed herein or a subsequence thereof, or a complement thereof. In a preferred embodiment, the promoter is a constitutive promoter.

In yet another embodiment, the present invention is directed to a transgenic plant/algae/cyanobacteria or plant/algae/cyanobacteria cells with improved plant/algae/cyanobacteria productivity, biomass production and/or photosynthesis, containing the nucleic acids described herein. In one aspect, plant/algae/cyanobacteria is Chlamydomonas reinhardtii, in another aspect, the microalgae is Ankistrodesmus, Botryococcus, Chlorella, Cyclotella, Dunaliella, Haematococcus, Nannochloropsis, Phaeodactylum, Porphyridium, Scenedesmus, Thalassiosira, or Volvox. Preferred plants/algae/cyanobacteria grown from the methods of the present invention include but are not limited to maize, Arabidopsis, soybean, sunflower, sorghum, canola, wheat, alfalfa, cotton, oat, rice, barley, tomato, cacao and millet. Plants/algae/cyanobacteria produced according to the invention can have at least one of the following phenotypes as compared to a non-modified control plant/algae/cyanobacteria, including but not limited to: increased dry weight, increased starch content, increased protein content, increased growth, as, for example measured by OD₇₅₀, increased plant height, increased root length, increased ear size, increased seed size, increased seed yield, or increased endosperm size when compared to a non-modified plant under similar conditions.

Detection of expression products is performed either qualitatively (by detecting presence or absence of one or more product of interest) or quantitatively (by monitoring the level of expression of one or more product of interest). Aspects of the invention optionally include monitoring an expression level or activity of a nucleic acid, polypeptide or chemical as noted herein for detection of the same in a plant/algae/cyanobacteria or in a population of plants/algae/cyanobacteria.

In a further aspect, the present invention relates to a polynucleotide amplified from a plant/algae nucleic acid library using primers which selectively hybridize, under stringent hybridization conditions, to loci within polynucleotides of the present invention.

Methods of the invention may be practiced using a number of known techniques, many of which are set forth below.

### Nucleic Acids

The present invention provides, inter alia, isolated nucleic acids of RNA, DNA, homologs, paralogs and orthologs and/or chimeras thereof, comprising LCI polynucleotides which function in a new plant/algae photosynthesis, biomass production, and productivity signaling pathway. This includes naturally occurring as well as synthetic variants and homologs of the sequences.

Sequences homologous, i.e., that share significant sequence identity or similarity, to those provided herein derived Arabidopsis thaliana or from other plants/algae of choice, are also an aspect of the invention. Homologous sequences can be derived from any plant/algae/cyanobacteria including monocots and dicots and in particular agriculturally important plant species, including but not limited to, crops such as soybean, wheat, corn (maize), potato, cotton, rice, rape, oilseed rape (including canola), sunflower, alfalfa, clover, sugarcane, and turf; or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, tomatillo, watermelon, rosaceous fruits (such as apple, peach, pear, cherry and plum) and vegetable brassicas (such as broccoli, cabbage, cauliflower, Brussels sprouts, and kohlrabi). Other crops, including fruits and vegetables, whose phenotype can be changed and which comprise homologous sequences include barley; rye; millet; sorghum; currant; avocado; citrus fruits such as oranges, lemons, grapefruit and tangerines, artichoke, cherries; nuts such as the walnut and peanut; endive; leek; roots such as arrowroot, beet, cassaya, turnip, radish, yam, and sweet potato; beans, and algae, including Archaeplastida such as Chlorophta (green algae), Rhodophyta (Red algae), Glaucophyta, Rhizaria, Excavata such as Chlorarachniophytes such as Euglenids, Chromista, Alveolata such as the Heterokonts, Bacillariophyceae (Diatoms), Axodine, Bolidomonas, Eustigmatophyceae, Phaeophyceae (Brown algae), Chrysophyceae (Golden algae), Raphidophyceae, Synurophyceae, Xanthophyceae (Yellow-green algae) Cryptophyta, Dinoflagellates, and Haptophyta. Particularly preferred is the green algae Clamydomonas. The homologous sequences may also be derived from woody species, such pine, poplar and eucalyptus, or mint or other labiates. In addition, homologous sequences may be derived from plants/algae that are evolutionarily-related to crop plants/algae, but which may not have yet been used as crop plants/algae. Examples include deadly nightshade (Atropa belladona), related to tomato; jimson weed (Datura strommium), related to peyote; and teosinte (Zea species), related to corn (maize).

### Orthologs and Paralogs

Homologous sequences as described above can comprise orthologous or paralogous sequences. Several different methods are known by those of skill in the art for identifying and defining these functionally homologous sequences. Three general methods for defining orthologs and paralogs are described; an ortholog, paralog or homolog may be identified by one or more of the methods described below.

Orthologs and paralogs are evolutionarily related genes that have similar sequence and similar functions. Orthologs are structurally related genes in different species that are derived by a speciation event. Paralogs are structurally related genes within a single species that are derived by a duplication event.

Within a single plant/algae species, gene duplication may cause two copies of a particular gene, giving rise to two or more genes with similar sequence and often similar function known as paralogs. A paralog is therefore a similar gene formed by duplication within the same species. Paralogs typically cluster together or in the same clade (a group of similar genes) when a gene family phylogeny is analyzed using programs such as CLUSTAL (Thompson et al. (1994) Nucleic Acids Res. 22: 4673-4680; Higgins et al. (1996) Methods Enzymol. 266: 383-402). Groups of similar genes can also be identified with pair-wise BLAST analysis (Feng and Doolittle (1987) J. Mol. Evol. 25: 351-360).

For example, a clade of very similar MADS domain transcription factors from Arabidopsis all share a common function in flowering time (Ratcliffe et al. (2001) Plant Physiol. 126: 122-132), and a group of very similar AP2 domain transcription factors from Arabidopsis are involved in tolerance of plants to freezing (Gilmour et al. (1998) Plant J. 16: 433-442). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount (2001), in Bioinformatics: Sequence and Genome Analysis Cold Spring Harbor Laboratory Press, Cold Spring Harbor, N.Y., page 543.)

Speciation, the production of new species from a parental species, can also give rise to two or more genes with similar sequence and similar function. These genes, termed orthologs, often have an identical function within their host plants/algae and are often interchangeable between species without losing function. Because plants/algae have common ancestors, many genes in any plant/algae species will have a corresponding orthologous gene in another plant/algae species. Once a phylogenic tree for a gene family of one species has been constructed using a program such as CLUSTAL (Thompson et al. (1994) Nucleic Acids Res. 22: 4673-4680; Higgins et al. (1996) supra) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

Orthologous genes from different organisms have highly conserved functions, and very often essentially identical functions (Lee et al. (2002) Genome Res. 12: 493-502; Remm et al. (2001) J. Mol. Biol. 314: 1041-1052). Paralogous genes, which have diverged through gene duplication, may retain similar functions of the encoded proteins. In such cases, paralogs can be used interchangeably with respect to certain embodiments of the instant invention (for example, transgenic expression of a coding sequence).

Variant Nucleotide Sequences in the Non-Coding Regions

The LCI polynucleotides (such as LCIA and/or LCIB) which function in the carbon transport pathway are used to generate variant nucleotide sequences having the nucleotide sequence of the 5′-untranslated region, 3′-untranslated region, or promoter region that is approximately 70%, 75%, 80%, 85%, 90% and 95% identical to the original nucleotide sequence of the corresponding sequences disclosed herein. These variants are then associated with natural variation in the germplasm for component traits related to and plant/algae photosynthesis, biomass production, and productivity. The associated variants are used as marker haplotypes to select for the desirable traits.

Variant Amino Acid Sequences of Polypeptides

Variant amino acid sequences of the LCI (such as LCIA and/or LCIB) polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using a protein alignment, an appropriate amino acid can be changed. Once the targeted amino acid is identified, the procedure outlined herein is followed. Variants having about 70%, 75%, 80%, 85%, 90% and 95% nucleic acid sequence identity are generated using this method. These variants are then associated with natural variation in the germplasm for component traits related to plant/algae photosynthesis, biomass production, and productivity. The associated variants are used as marker haplotypes to select for the desirable traits.

The present invention also includes polynucleotides optimized for expression in different organisms. For example, for expression of the polynucleotide in a maize plant, the sequence can be altered to account for specific codon preferences and to alter GC content as according to Murray, et al, supra. Maize codon usage for 28 genes from maize plants/algae is listed in Table 4 of Murray, et al., supra.

The LCI polynucleotides (such as LCIA and/or LCIB) comprise isolated polynucleotides which are inclusive of:
(a) a polynucleotide encoding a LCI polypeptide and conservatively modified and polymorphic variants thereof;
(b) a polynucleotide having at least 70% sequence identity with polynucleotides of (a);
(c) complementary sequences of polynucleotides of (a) or (b).

### Construction of Nucleic Acids

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a fungus or bacteria.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. The nucleic acid of the present invention—excluding the polynucleotide sequence—is optionally a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3′SS, pGEM, pSK+/−, pGEX, pSPORTI and II, pOPRSVI CAT, pOPI3 CAT, pXT1, pSG5, pPbac, pMbac, pMC1neo, pOG44, pOG45, pFRTβGAL, pNEOβGAL, pRS403, pRS404, pRS405, pRS406, pRS413, pRS414, pRS415, pRS416, lambda MOSSlox, and lambda MOSElox. Optional vectors for the present invention, include but are not limited to, lambda ZAP II, and pGEX. For a description of various nucleic acids see, e.g., Stratagene Cloning Systems, Catalogs 1995, 1996, 1997 (La Jolla, Calif.); and, Amersham Life Sciences, Inc., Catalog '97 (Arlington Heights, Ill.).

### Synthetic Methods for Constructing Nucleic Acids

The isolated nucleic acids of the present invention can also be prepared by direct chemical synthesis by methods such as the phosphotriester method of Narang, et al., (1979) Meth. Enzymol. 68:90-9; the phosphodiester method of Brown, et al., (1979) Meth. Enzymol. 68:109-51; the diethylphosphoramidite method of Beaucage, et al., (1981) Tetra. Letts. 22(20):1859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68; and, the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G>7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present invention provides 5′ and/or 3′ UTR regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present invention can be analyzed statistically using commercially available software packages such as “Codon Preference” available from the University of Wisconsin Genetics Computer Group. See, Devereaux, et al., (1984) Nucleic Acids Res. 12:387-395); or MacVector 4.1 (Eastman Kodak Co., New Haven, Conn.). Thus, the present invention provides a codon usage frequency characteristic of the coding region of at least one of the polynucleotides of the present invention. The number of polynucleotides (3 nucleotides per amino acid) that can be used to determine a codon usage frequency can be any integer from 3 to the number of polynucleotides of the present invention as provided herein. Optionally, the polynucleotides will be full-length sequences. An exemplary number of sequences for statistical analysis can be at least 1, 5, 10, 20, 50 or 100.

### Sequence Shuffling

The present invention provides methods for sequence shuffling using polynucleotides of the present invention, and compositions resulting therefrom. Sequence shuffling is described in PCT publication No. 96/19256. See also, Zhang, et al., (1997) Proc. Natl. Acad. Sci. USA 94:4504-9; and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present invention further provides recombinant expression cassettes comprising a nucleic acid of the present invention. A nucleic acid sequence coding for the desired polynucleotide of the present invention, for example a cDNA or a genomic sequence encoding a polypeptide long enough to code for an active protein of the present invention, can be used to construct a recombinant expression cassette which can be introduced into the desired host cell. A recombinant expression cassette will typically comprise a polynucleotide of the present invention operably linked to transcriptional initiation regulatory sequences which will direct the transcription of the polynucleotide in the intended host cell, such as tissues of a transformed plant/algae.

For example, plant/algae expression vectors may include (1) a cloned plant/algae gene under the transcriptional control of 5′ and 3′ regulatory sequences and (2) a dominant selectable marker. Such plant/algae expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A plant/algae promoter fragment can be employed which will direct expression of a polynucleotide of the present invention in all tissues of a regenerated plant/algae. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30); and maize H3 histone (Lepetit, et al., (1992) Mol. Gen. Genet. 231:276-85; and Atanassvoa, et al., (1992) Plant Journal 2(3):291-300); ALS promoter, as described in PCT Application No. WO 96/30530; and other transcription initiation regions from various plant/algae genes known to those of skill in the art.

Alternatively, the plant/algae promoter can direct expression of a polynucleotide of the present invention in a specific tissue or may be otherwise under more precise environmental or developmental control. Such promoters are referred to here as “inducible” promoters. Environmental conditions that may affect transcription by inducible promoters include pathogen attack, anaerobic conditions, or the presence of light. Examples of inducible promoters are the Adh1 promoter, which is inducible by hypoxia or cold stress, the Hsp70 promoter, which is inducible by heat stress, and the PPDK promoter, which is inducible by light.

Examples of promoters under developmental control include promoters that initiate transcription only, or preferentially, in certain tissues, such as leaves, roots, fruit, seeds, or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant/algae genes, or from T-DNA. The 3′ end sequence to be added can be derived from, for example, the nopaline synthase or octopine synthase genes, or alternatively from another plant/algae gene, or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50; and An, et al., (1989) Plant Cell 1:115-22); and the CaMV 19S gene (Mogen, et al., (1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant/algae and animal expression constructs has been shown to increase gene expression at both the mRNA and protein levels up to 1000-fold (Buchman and Berg, (1988) Mol. Cell. Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, The Maize Handbook, Chapter 116, Freeling and Walbot, eds., Springer, N.Y. (1994).

Plant/algae signal sequences, including, but not limited to, signal-peptide encoding DNA/RNA sequences which target proteins to the extracellular matrix of the plant/algae cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant/algae Cell, 2:301-13); signal peptides which cause proteins to be secreted, such as that of PRIb (Lind, et al., (1992) Plant Mol. Biol. 18:47-53) or the barley alpha amylase (BAA) (Rahmatullah, et al., (1989) Plant Mol. Biol. 12:119, and hereby incorporated by reference), or signal peptides which target proteins to the plastids such as that of rapeseed enoyl-Acp reductase (Verwaert, et al., (1994) Plant Mol. Biol. 26:189-202) are useful in the invention.

The vector comprising the sequences from a polynucleotide of the present invention will typically comprise a marker gene, which confers a selectable phenotype on plant/algae cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene), or other such genes known in the art. The bar gene encodes resistance to the herbicide basta, and the ALS gene encodes resistance to the herbicide chlorsulfuron.

Typical vectors useful for expression of genes in higher plants/algae are well known in the art and include vectors derived from the tumor-inducing (Ti) plasmid of Agrobacterium tumefaciens described by Rogers, et al. (1987), Meth. Enzymol. 153:253-77. These vectors are plant/algae integrating vectors in that on transformation, the vectors integrate a portion of vector DNA into the genome of the host plant/algae. Exemplary A. tumefaciens vectors useful herein are plasmids pKYLX6 and pKYLX7 of Schardl, et al., (1987) Gene 61:1-11, and Berger, et al., (1989) Proc. Natl. Acad. Sci. USA, 86:8402-6. Another useful vector herein is plasmid pBI101.2 that is available from CLONTECH Laboratories, Inc. (Palo Alto, Calif.).

### Expression of Proteins in Host Cells

Using the nucleic acids of the present invention, one may express a protein of the present invention in a recombinantly engineered cell such as bacteria, yeast, insect, mammalian, or preferably plant/algae cells. The cells produce the protein in a non-natural condition (e.g., in quantity, composition, location, and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high level expression of a cloned gene, it is desirable to construct expression vectors which contain, at the minimum, a strong promoter, such as ubiquitin, to direct transcription, a ribosome binding site for translational initiation, and a transcription/translation terminator. Constitutive promoters are classified as providing for a range of constitutive expression. Thus, some are weak constitutive promoters, and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level,” or about 1/10 transcripts to about 1/100 transcripts to about 1/1,000 transcripts.

One of skill would recognize that modifications could be made to a protein of the present invention without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly H is) placed on either terminus to create conveniently located restriction sites or termination codons or purification sequences.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline, or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present invention.

### Expression in Eukaryotes

A variety of eukaryotic expression systems such as yeast, insect cell lines, plant/algae and mammalian cells, are known to those of skill in the art. As explained briefly below, the present invention can be expressed in these eukaryotic systems. In some embodiments, transformed/transfected plant/algae cells, as discussed infra, are employed as expression systems for production of the proteins of the instant invention.

Synthesis of heterologous proteins in yeast is well known. Sherman, et al., (1982) Methods in Yeast Genetics, Cold Spring Harbor Laboratory is a well-recognized work describing the various methods available to produce the protein in yeast. Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present invention can also be ligated to various expression vectors for use in transfecting cell cultures of, for instance, mammalian, insect, or plant/algae origin. Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49), and necessary processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection Catalogue of Cell Lines and Hybridomas (7^{th} ed., 1992).

Appropriate vectors for expressing proteins of the present invention in insect cells are usually derived from the SF9 baculovirus. Suitable insect cell lines include mosquito larvae, silkworm, armyworm, moth, and Drosophila cell lines such as a Schneider cell line (see, e.g., Schneider, (1987) J. Embryol. Exp. Morphol. 27:353-65).

As with yeast, when higher animal or plant/algae host cells are employed, polyadenlyation or transcription terminator sequences are typically incorporated into the vector. An example of a terminator sequence is the polyadenlyation sequence from the bovine photosynthesis, biomass production, and productivity hormone gene. Sequences for accurate splicing of the transcript may also be included. An example of a splicing sequence is the VP1 intron from SV40 (Sprague et al., J. Virol. 45:773-81 (1983)). Additionally, gene sequences to control replication in the host cell may be incorporated into the vector such as those found in bovine papilloma virus type-vectors (Saveria-Campo, “Bovine Papilloma Virus DNA a Eukaryotic Cloning Vector,” in DNA Cloning: A Practical Approach, vol. II, Glover, ed., IRL Press, Arlington, Va., pp. 213-38 (1985)).

In addition, the LCI gene placed in the appropriate plant/algae expression vector can be used to transform plant/algae cells. The polypeptide can then be isolated from plant/algae callus or the transformed cells can be used to regenerate transgenic plants/algae. Such transgenic plants/algae can be harvested, and the appropriate tissues (seed or leaves, for example) can be subjected to large scale protein extraction and purification techniques.

### Plant/Algae Transformation Methods

Numerous methods for introducing foreign genes into plants/algae are known and can be used to insert LCI polynucleotides which function in applicant's plant/algae photosynthesis, biomass production, and productivity pathway into a plant/algae host, including biological and physical plant/algae transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant/algae, and include chemical transfection methods such as calcium phosphate, microorganism-mediated gene transfer such as Agrobacterium (Horsch et al., Science 227:1229-31 (1985)), electroporation, micro-injection, and biolistic bombardment.

Expression cassettes and vectors and in vitro culture methods for plant/algae cell or tissue transformation and regeneration of plants are known and available. See, e.g., Gruber et al., “Vectors for Plant Transformation,” in Methods in Plant Molecular Biology and Biotechnology, supra, pp. 89-119.

The isolated polynucleotides or polypeptides may be introduced into the plant/algae by one or more techniques typically used for direct delivery into cells. Such protocols may vary depending on the type of organism, cell, plant/algae or plant/algae cell, i.e. monocot or dicot, targeted for gene modification. Suitable methods of transforming plant/algae cells include microinjection (Crossway, et al., (1986) Biotechniques 4:320-334; and U.S. Pat. No. 6,300,543), electroporation (Riggs, et al., (1986) Proc. Natl. Acad. Sci. USA 83:5602-5606, direct gene transfer (Paszkowski et al., (1984) EMBO J. 3:2717-2722), and ballistic particle acceleration (see, for example, Sanford, et al., U.S. Pat. No. 4,945,050; WO 91/10725; and McCabe, et al., (1988) Biotechnology 6:923-926). Also see, Tomes, et al., “Direct DNA Transfer into Intact Plant Cells Via Microprojectile Bombardment”. pp. 197-213 in Plant Cell, Tissue and Organ Culture, Fundamental Methods. eds. O. L. Gamborg & G. C. Phillips. Springer-Verlag Berlin Heidelberg N.Y., 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 91/10725 (maize); Klein, et al., (1988) Plant Physiol. 91:440-444 (maize); Fromm, et al., (1990) Biotechnology 8:833-839; and Gordon-Kamm, et al., (1990) Plant Cell 2:603-618 (maize); Hooydaas-Van Slogteren & Hooykaas (1984) Nature (London) 311:763-764; Bytebierm, et al., (1987) Proc. Natl. Acad. Sci. USA 84:5345-5349 (Liliaceae); De Wet, et al., (1985) In The Experimental Manipulation of Ovule Tissues, ed. G. P. Chapman, et al., pp. 197-209. Longman, N.Y. (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255; and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185); all of which are herein incorporated by reference.

### Agrobacterium-Mediated Transformation

The most widely utilized method for introducing an expression vector into plants/algae is based on the natural transformation system of Agrobacterium. A. tumefaciens and A. rhizogenes are plant/algae pathogenic soil bacteria, which genetically transform plant/algae cells. The Ti and Ri plasmids of A. tumefaciens and A. rhizogenes, respectively, carry genes responsible for genetic transformation of plants/algae. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra; and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants/algae. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993; and Simpson, et al., (1986) Plant Mol. Biol. 6:403-15 (also referenced in the '306 patent); all incorporated by reference in their entirety.

Once constructed, these plasmids can be placed into A. rhizogenes or A. tumefaciens and these vectors used to transform cells of plant/algae species, which are ordinarily susceptible to Fusarium or Alternaria infection. Several other transgenic plants/algae are also contemplated by the present invention. The selection of either A. tumefaciens or A. rhizogenes will depend on the plant/algae being transformed thereby. In general A. tumefaciens is the preferred organism for transformation. Most dicotyledonous plants, algae, some gymnosperms, and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae, and Chenopodiaceae. Monocot plants can now be transformed with some success. European Patent Application No. 604 662 A1 discloses a method for transforming monocots using Agrobacterium. European Application No. 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants/algae. For example, whole plants/algae can be infected with these vectors by wounding the plant/algae and then introducing the vector into the wound site. Any part of the plant/algae can be wounded, including leaves, stems and roots. Alternatively, plant/algae tissue, in the form of an explant, such as cotyledonary tissue or leaf disks, can be inoculated with these vectors, and cultured under conditions, which promote plant/algae regeneration. Roots or shoots transformed by inoculation of plant/algae tissue with A. rhizogenes or A. tumefaciens, containing the gene coding for the fumonisin degradation enzyme, can be used as a source of plant/algae tissue to regenerate fumonisin-resistant transgenic plants/algae, either via somatic embryogenesis or organogenesis. Examples of such methods for regenerating plant/algae tissue are disclosed in Shahin, (1985) Theor. Appl. Genet. 69:235-40; U.S. Pat. No. 4,658,082; Simpson, et al., supra; and U.S. patent application Ser. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant/algae transformation, collectively referred to as direct gene transfer, have been developed as an alternative to Agrobacterium-mediated transformation.

A generally applicable method of plant/algae transformation is microprojectile-mediated transformation, where DNA is carried on the surface of microprojectiles measuring about 1 to 4 μm. The expression vector is introduced into plant/algae tissues with a biolistic device that accelerates the microprojectiles to speeds of 300 to 600 m/s which is sufficient to penetrate the plant/algae cell walls and membranes (Sanford, et al., (1987) Part. Sci. Technol. 5:27; Sanford, (1988) Trends Biotech 6:299; Sanford, (1990) Physiol. Plant 79:206; and Klein, et al., (1992) Biotechnology 10:268).

Another method for physical delivery of DNA to plants/algae is sonication of target cells as described in Zang, et al., (1991) BioTechnology 9:996. Alternatively, liposome or spheroplast fusions have been used to introduce expression vectors into plants/algae. See, e.g., Deshayes, et al., (1985) EMBO J. 4:2731; and Christou, et al., (1987) Proc. Natl. Acad. Sci. USA 84:3962. Direct uptake of DNA into protoplasts using CaCl₂ precipitation, polyvinyl alcohol, or poly-L-ornithine has also been reported. See, e.g., Hain, et al., (1985) Mol. Gen. Genet. 199:161; and Draper, et al., (1982) Plant Cell Physiol. 23:451.

Electroporation of protoplasts and whole cells and tissues has also been described. See, e.g., Donn, et al., (1990) Abstracts of the VIIth Intl. Congress on Plant Cell and Tissue Culture IAPTC, A2-38, p. 53; D'Halluin, et al., (1992) Plant Cell 4:1495-505; and Spencer, et al., (1994) Plant Mol. Biol. 24:51-61.

Increasing the Activity and/or Level of a LCI Polypeptide

Methods are provided to increase the activity and/or level of the LCI (such as LCIA or LCIB) polypeptides to increase plant/algae photosynthesis, biomass production, and productivity in a high CO₂ environment. An increase in the level and/or activity of the LCI (such as LCIA or LCIB) polypeptide can be achieved by providing to the plant/algae a LCI (such as LCIA or LCIB) polypeptide. The LCI (such as LCIA or LCIB) polypeptide can be provided by introducing the amino acid sequence encoding the LCI (such as LCIA or LCIB) polypeptide into the plant/algae, introducing into the plant/algae a nucleotide sequence encoding a LCI (such as LCIA or LCIB) polypeptide or alternatively by modifying a genomic locus encoding the LCI (such as LCIA or LCIB) polypeptide.

As discussed elsewhere herein, many methods are known the art for providing a polypeptide to a plant/algae including, but not limited to, direct introduction of the polypeptide into the plant/algae, introducing into the plant/algae (transiently or stably) a polynucleotide construct encoding a polypeptide having LCI activity. It is also recognized that the methods of the invention may employ a polynucleotide that is not capable of directing, in the transformed plant/algae, the expression of a protein or an RNA. Thus, the level and/or activity of a LCI (such as LCIA or LCIB) polypeptide may be increased by altering the gene encoding the LCI (such as LCIA or LCIB) polypeptide or its promoter. See, e.g., Kmiec, U.S. Pat. No. 5,565,350; Zarling, et al., PCT/US93/03868. Therefore mutagenized plants/algae that carry mutations in LCI (such as LCIA or LCIB) genes, where the mutations increase expression of the LCI (such as LCIA or LCIB) gene or increase the LCI (such as LCIA or LCIB) activity of the encoded LCI (such as LCIA or LCIB) polypeptide are provided.

Reducing the Activity and/or Level of a LCI (such as LCIA or lcib) Polypeptide

In some embodiments, methods may be provided to reduce or eliminate the activity of a LCI (such as LCIA or lcib) polypeptide of the invention by transforming a plant/algae cell with an expression cassette that expresses a polynucleotide that inhibits the expression of the LCI (such as LCIA or lcib) polypeptide. The polynucleotide may inhibit the expression of the LCI (such as LCIA or lcib) polypeptide directly, by preventing transcription or translation of the LCI (such as LCIA or lcib) messenger RNA, or indirectly, by encoding a polypeptide that inhibits the transcription or translation of an LCI (such as LCIA or lcib) gene encoding LCI (such as LCIA or lcib) polypeptide. Methods for inhibiting or eliminating the expression of a gene in a plant/algae are well known in the art, and any such method may be used in the present invention to inhibit the expression of LCI (such as LCIA or lcib) polypeptide.

In accordance with the present invention, the expression of LCI (such as LCIA or lcib) polypeptide is inhibited if the protein level of the LCI (such as LCIA or lcib) polypeptide is less than 70% of the protein level of the same LCI (such as LCIA or lcib) polypeptide in a plant/algae that has not been genetically modified or mutagenized to inhibit the expression of that LCI (such as LCIA or lcib) polypeptide. In particular embodiments of the invention, the protein level of the LCI (such as LCIA or lcib) polypeptide in a modified plant/algae according to the invention is less than 60%, less than 50%, less than 40%, less than 30%, less than 20%, less than 10%, less than 5%, or less than 2% of the protein level of the same LCI (such as LCIA or lcib) polypeptide in a plant/algae that is not a mutant or that has not been genetically modified to inhibit the expression of that LCI (such as LCIA or lcib) polypeptide. The expression level of the LCI (such as LCIA or lcib) polypeptide may be measured directly, for example, by assaying for the level of LCI (such as LCIA or lcib) polypeptide expressed in the plant/algae cell or plant/algae, or indirectly, for example, by measuring the phenotypic changes in the plant/algae. Methods for performing such assays are described elsewhere herein.

In other embodiments of the invention, the activity of the LCI (such as LCIA or lcib) polypeptide is reduced or eliminated by transforming a plant/algae cell with an expression cassette comprising a polynucleotide encoding a polypeptide that inhibits the activity of a LCI (such as LCIA or lcib) polypeptide. The LCI (such as LCIA or lcib) activity of a LCI (such as LCIA or lcib) polypeptide is inhibited according to the present invention if the activity of the LCI (such as LCIA or lcib) polypeptide is less than 70% of the activity of the same LCI (such as LCIA or lcib) polypeptide in a plant/algae that has not been modified to inhibit the LCI (such as LCIA or lcib) activity of that polypeptide. In particular embodiments of the invention, the LCI (such as LCIA or lcib) activity of the LCI (such as LCIA or lcib) polypeptide in a modified plant/algae according to the invention is less than 60%, less than 50%, less than 40%, less than 30%, less than 20%, less than 10%, or less than 5% of the LCI (such as LCIA or lcib) activity of the same polypeptide in a plant/algae that that has not been modified to inhibit the expression of that LCI (such as LCIA or lcib) polypeptide. The LCI (such as LCIA or lcib) activity of a LCI (such as LCIA or lcib) polypeptide is “eliminated” according to the invention when it is not detectable by the assay methods described elsewhere herein. Methods of determining the alteration of activity of a LCI (such as LCIA or lcib) polypeptide are described elsewhere herein.

In other embodiments, the activity of a LCI (such as LCIA or lcib) polypeptide may be reduced or eliminated by disrupting the gene encoding the LCI (such as LCIA or lcib) polypeptide. The invention encompasses mutagenized plants/algae that carry T-DNA insertions or mutations in LCI (such as LCIA or lcib) genes, where the mutations reduce expression of the LCI (such as LCIA or lcib) gene or inhibit the activity of the encoded LCI (such as LCIA or lcib) polypeptide.

Thus, many methods may be used to reduce or eliminate the activity of a LCI (such as LCIA or lcib) polypeptide. In addition, more than one method may be used to reduce the activity of a single LCI (such as LCIA or lcib) polypeptide.

The following examples are intended for illustration purposes only and are not intended to limit the invention in any way.

### EXAMPLES

### Example 1

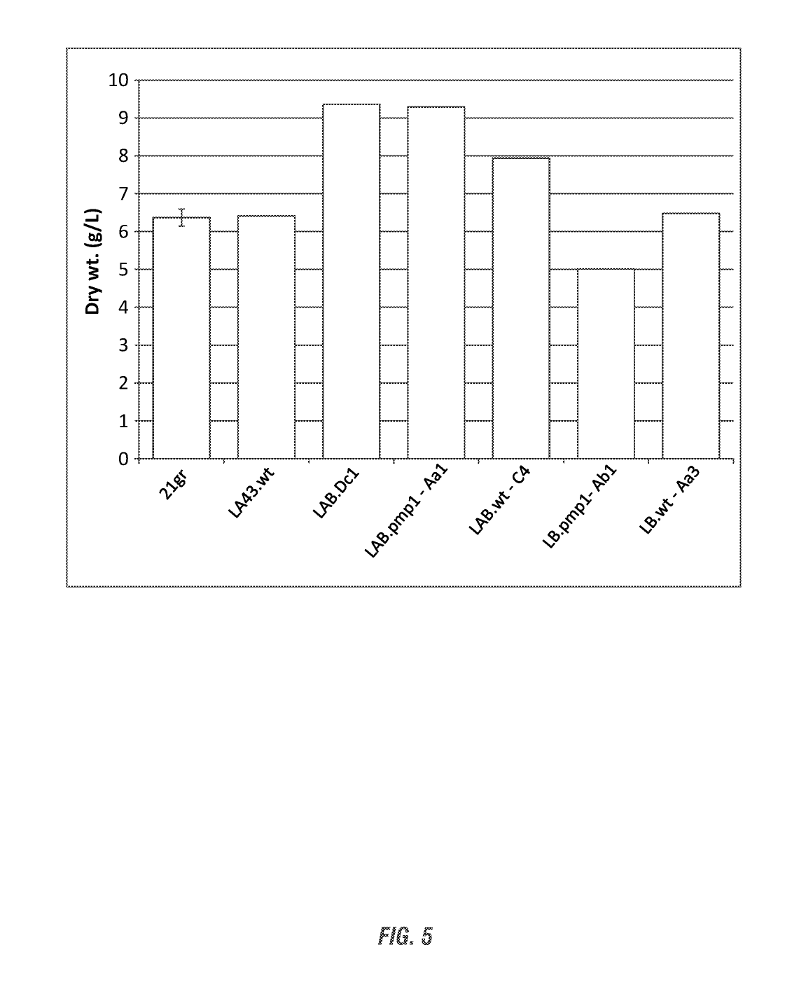

Methodology used herein can generally be found in Wang, et. al, PNAS Jun. 27, 2006 vol. 103, no. 25; pages 10110-10115, “An Inorganic Carbon Transport System Responsible for Acclimation Specific to Air Levels of CO₂ in Chlamydomonas reinhardtii” which is hereby expressly incorporated by reference in its entirety particularly pages 10114-10115 and FIG. 5 in the Materials and Methods section.

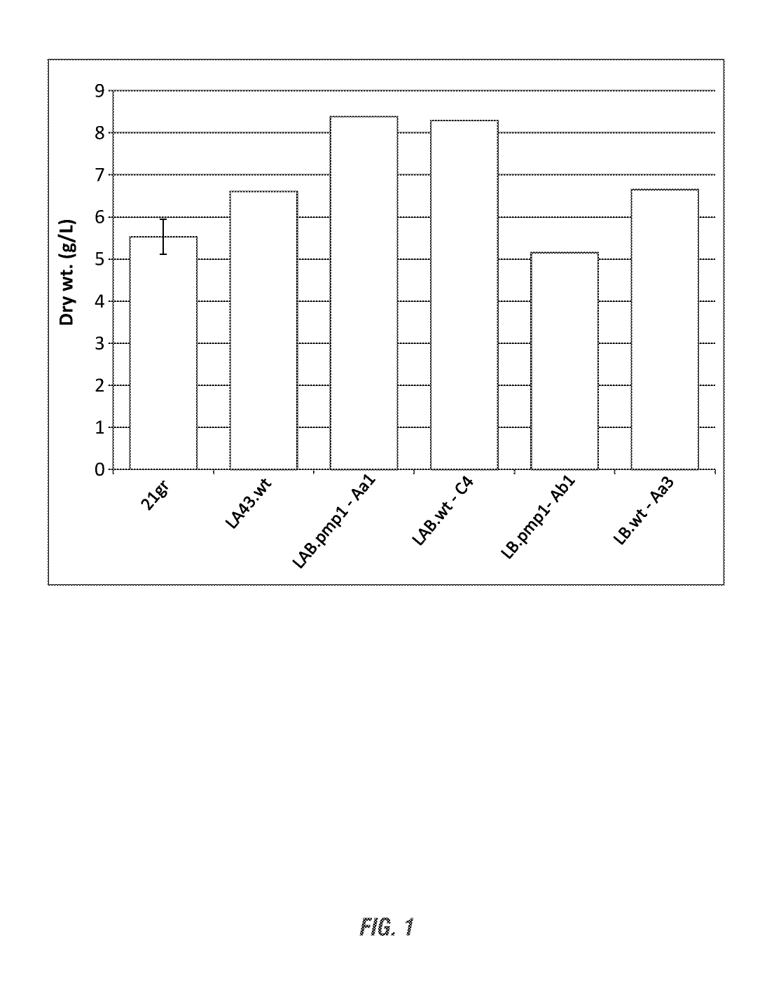

FIG. 1. A transgenic Chlamydomonas lcib mutant line expressing an inserted lcib gene regulated by a high expression, constitutive promoter was crossed with a transgenic Chlamydomonas line expressing an inserted LCIA gene regulated by a high expression, constitutive promoter. Progeny from this cross with different genotypes, including the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background (LAB.wt-C4) and the LCIA and LCIB transgenes both in an lcib mutant background (LAB.pmp1-Aa1), were grown along with WT (21gr) in 200 ml photobioreactors to stationary phase, then harvested by centrifugation and the cell pellets dried for biomass determination. Values for each strain represent the average from two separate cultures in different mini-bioreactors, except that for 21gr, which represents the average of several separate cultures.

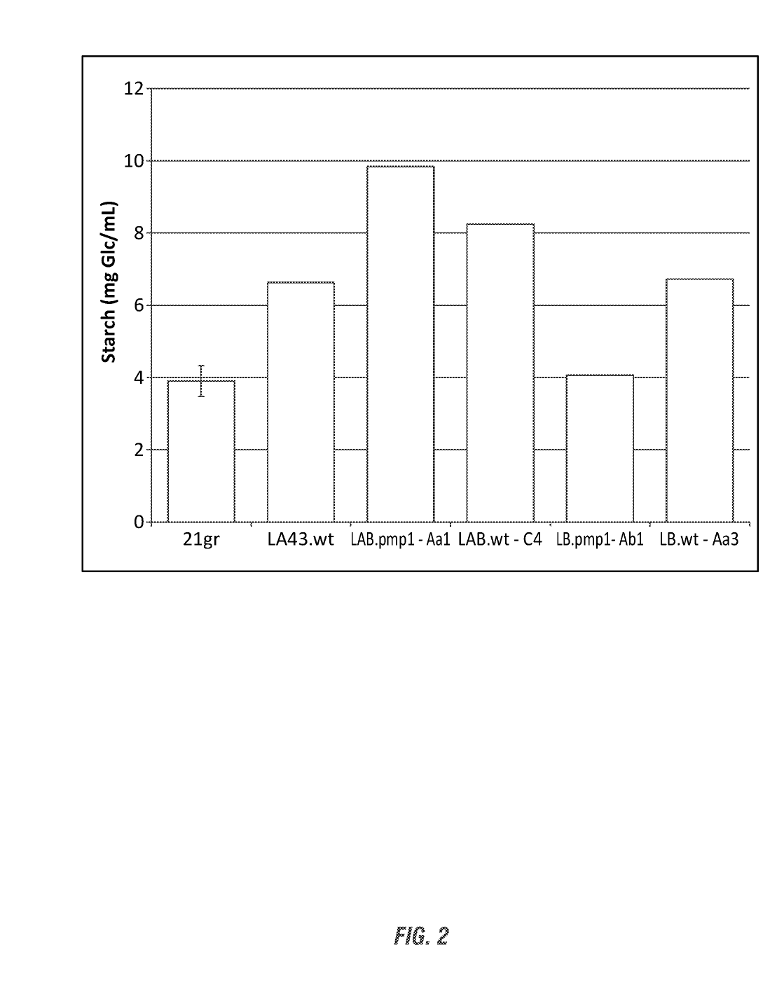

FIG. 2. Additional data from experiment illustrated in FIG. 1. Starch accumulation in wild-type and transgenic Chlamydomonas strains grown to stationary phase in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background (LAB.wt-C4) and the LCIA and LCIB transgenes both in an lcib mutant background (LAB.pmp1-Aa1). Samples were removed just prior to the final harvest, centrifuged and the cell pellets assayed for starch content by determining glucose (Sigma GAHK-20 kit) after starch digestion with amyloglucosidase. Values for each strain represent the average from two separate cultures in different mini-bioreactors, except that for 21gr, which represents the average of several separate cultures.

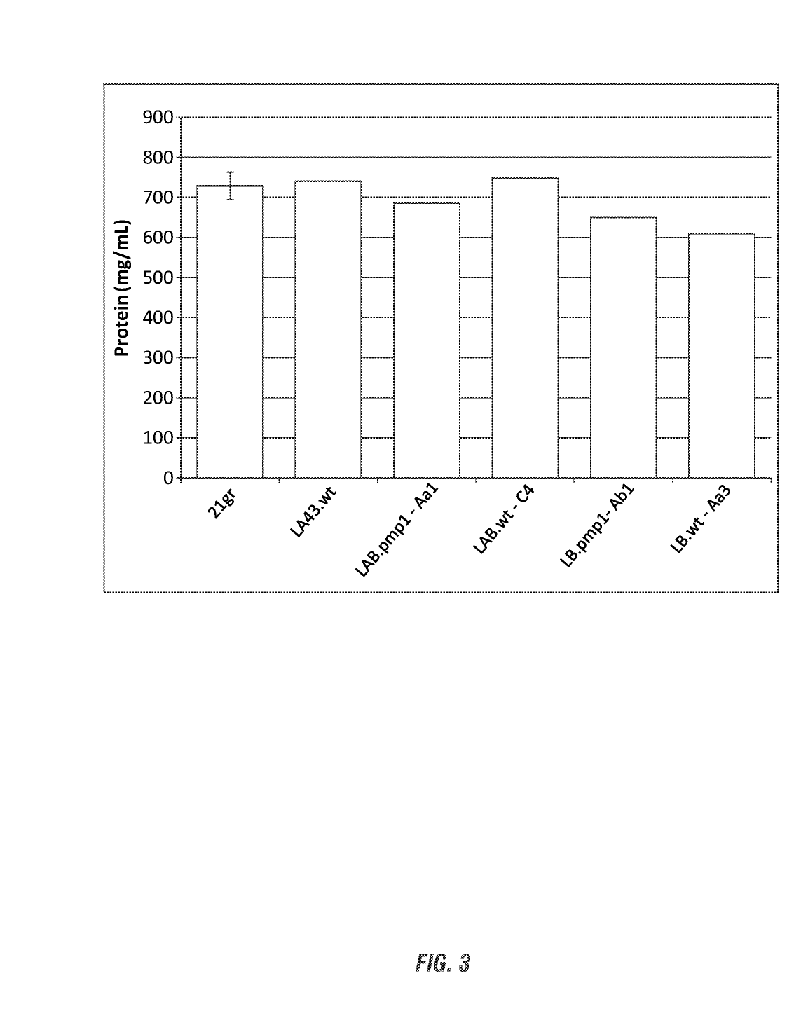

FIG. 3. Additional data from experiment illustrated in FIG. 1. Protein content of wild-type and transgenic Chlamydomonas strains grown to stationary phase in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background (LAB.wt-C4) and the LCIA and LCIB transgenes both in an lcib mutant background (LAB.pmp1-Aa1). Samples were removed just prior to the final harvest, centrifuged and the cell pellets assayed for total protein content using the Bio-Rad Protein Assay (Bio-Rad Laboratories, Hercules, Calif.). Values for each strain represent the average from two separate cultures in different mini-bioreactors, except that for 21gr, which represents the average of several separate cultures.

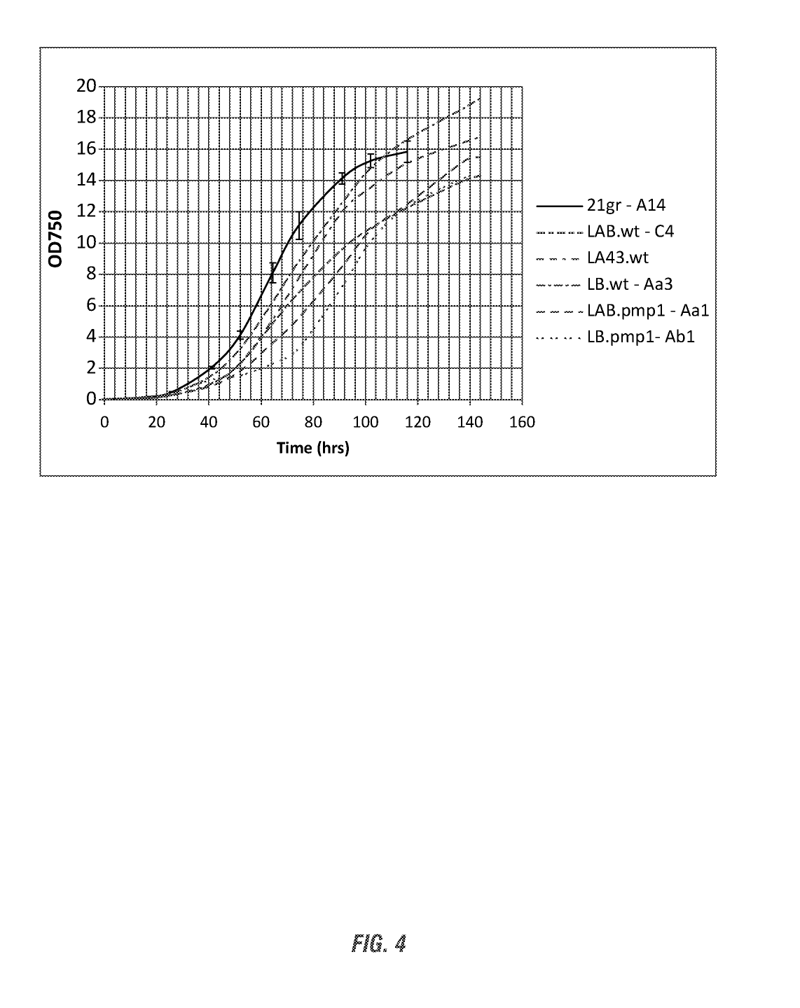

FIG. 4. Additional data from experiment illustrated in FIG. 2. Cell density estimates for wild-type and transgenic Chlamydomonas strains determined over the course of culture growth in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background in two independent progeny (LAB.wt-C4 and LAB.Dc1) and the LCIA and LCIB transgenes both in an lcib mutant background (LAB.pmp1-Aa1). Samples were removed at frequent intervals, diluted into a measurable range and the OD₇₅₀ determined in a UV/Vis spectrophotometer. Values for each strain represent the average from two separate cultures in different mini-bioreactors, except that for 21gr, which represents the average of several separate cultures.

FIG. 5. Replication of experiment illustrated in FIG. 1. A transgenic Chlamydomonas lcib mutant line expressing an inserted LCIB gene regulated by a high expression, constitutive promoter was crossed with a transgenic Chlamydomonas line expressing an inserted LCIA gene regulated by a high expression, constitutive promoter. Progeny from this cross with different genotypes, including the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background in two independent progeny (LAB.wt-C4 and LAB.Dc1) and the LCIA and LCIB transgenes both in an lcib mutant background (LAB.pmp1-Aa1), were grown along with WT (21gr) in 200 ml photobioreactors to stationary phase, then harvested by centrifugation and the cell pellets dried for biomass determination. Values for each strain represent the average from two separate cultures in different mini-bioreactors, except that for 21gr, which represents the average of several separate cultures.

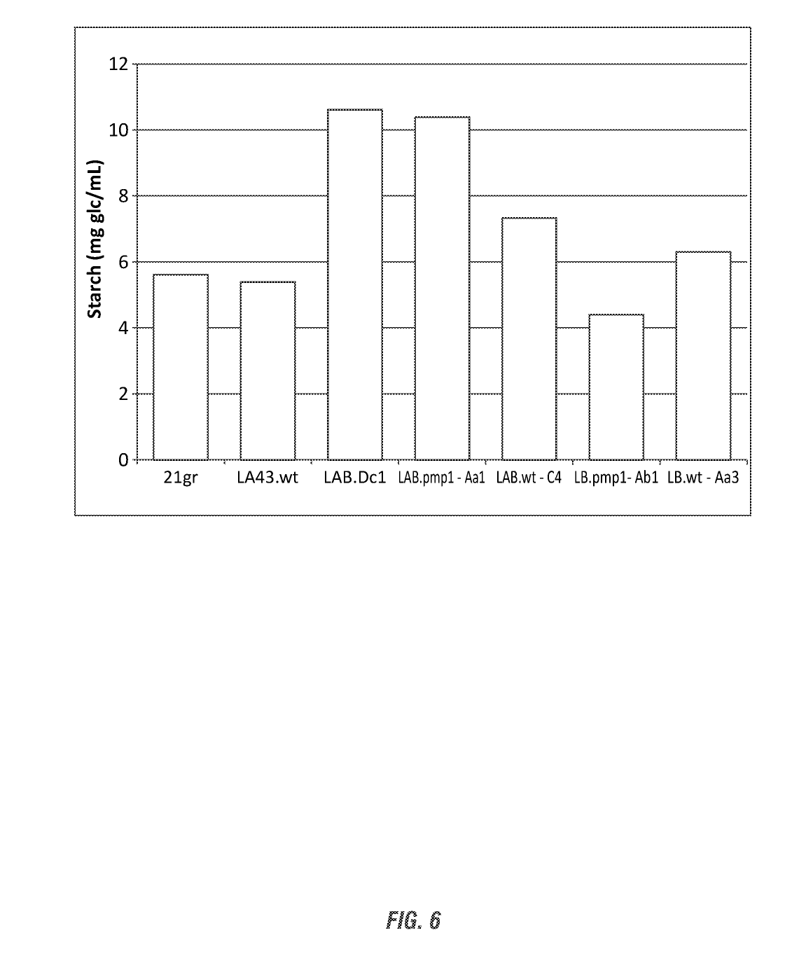

FIG. 6. Additional data from experiment illustrated in FIG. 5. Starch accumulation in wild-type and transgenic Chlamydomonas strains grown to stationary phase in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background in two independent progeny (LAB.wt-C4 and LAB.Dc1) and the LCIA and LCIB transgenes both in an lcib mutant background (LAB.pmp1-Aa1). Samples were removed just prior to the final harvest, centrifuged and the cell pellets assayed for starch content by determining glucose (Sigma GAHK-20 kit) after starch digestion with amyloglucosidase. Values for each strain represent the average from two separate cultures in different mini-bioreactors, except that for 21gr, which represents the average of several separate cultures.

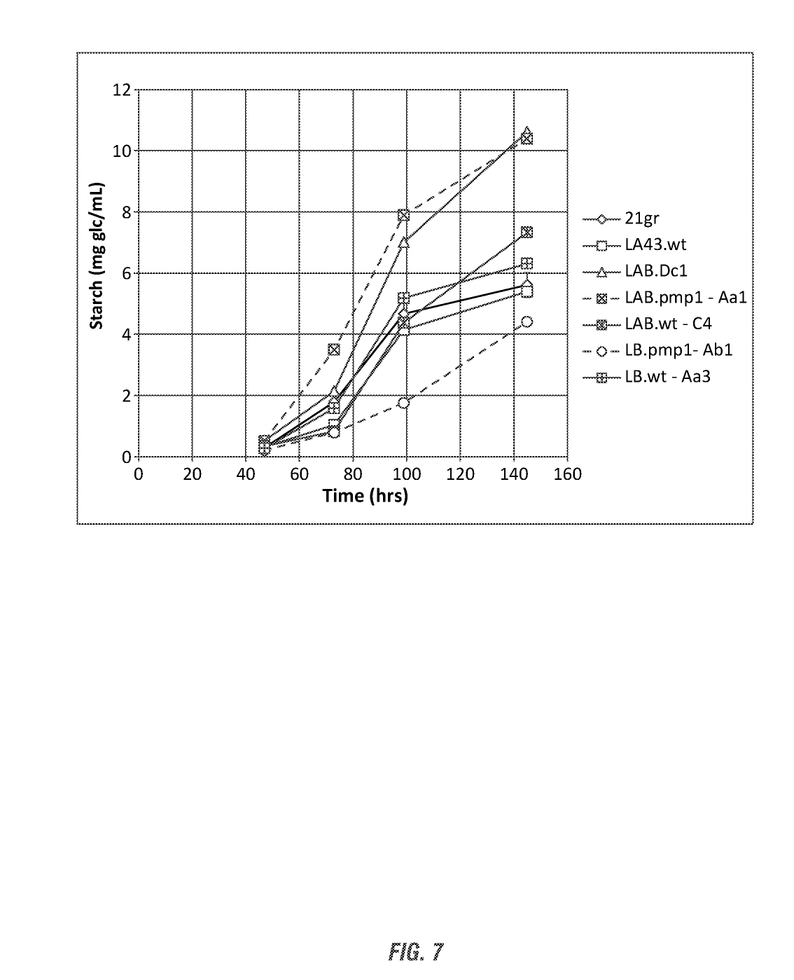

FIG. 7. Additional data from experiment illustrated in FIG. 5. Starch accumulation in wild-type and transgenic Chlamydomonas strains determined over the course of culture growth in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background in two independent progeny (LAB.wt-C4 and LAB.Dc1) and the LCIA and LCIB transgenes both in an lcib mutant background (LAB.pmp1-Aa1). Samples were removed at indicated intervals, centrifuged and the cell pellets assayed for starch content by determining glucose (Sigma GAHK-20 kit) after starch digestion with amyloglucosidase. Values for each strain represent the average from two separate cultures in different mini-bioreactors, except that for 21gr, which represents the average of several separate cultures.

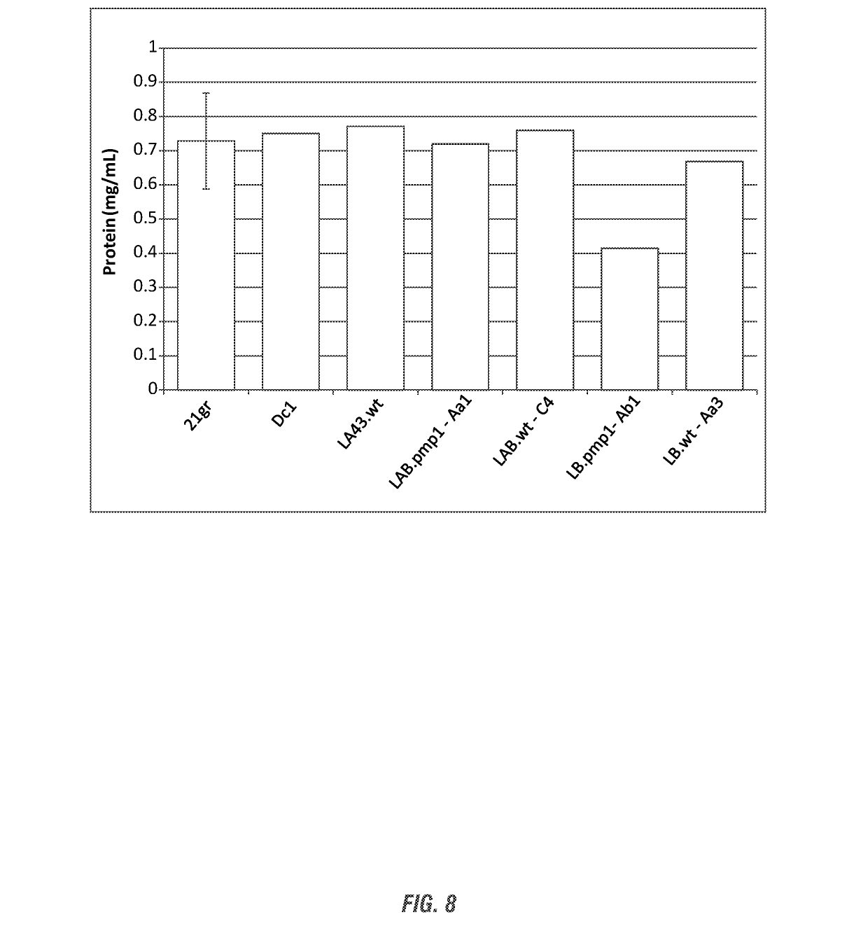

FIG. 8. Additional data from experiment illustrated in FIG. 5. Protein content of wild-type and transgenic Chlamydomonas strains grown to stationary phase in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background in two independent progeny (LAB.wt-C4 and LAB.Dc1) and the LCIA and LCIB transgenes both in an lcib mutant background (LAB.pmp1-Aa1). Samples were removed just prior to the final harvest, centrifuged and the cell pellets assayed for total protein content using the Bio-Rad Protein Assay (Bio-Rad Laboratories, Hercules, Calif.). Values for each strain represent the average from two separate cultures in different mini-bioreactors, except that for 21gr, which represents the average of several separate cultures.

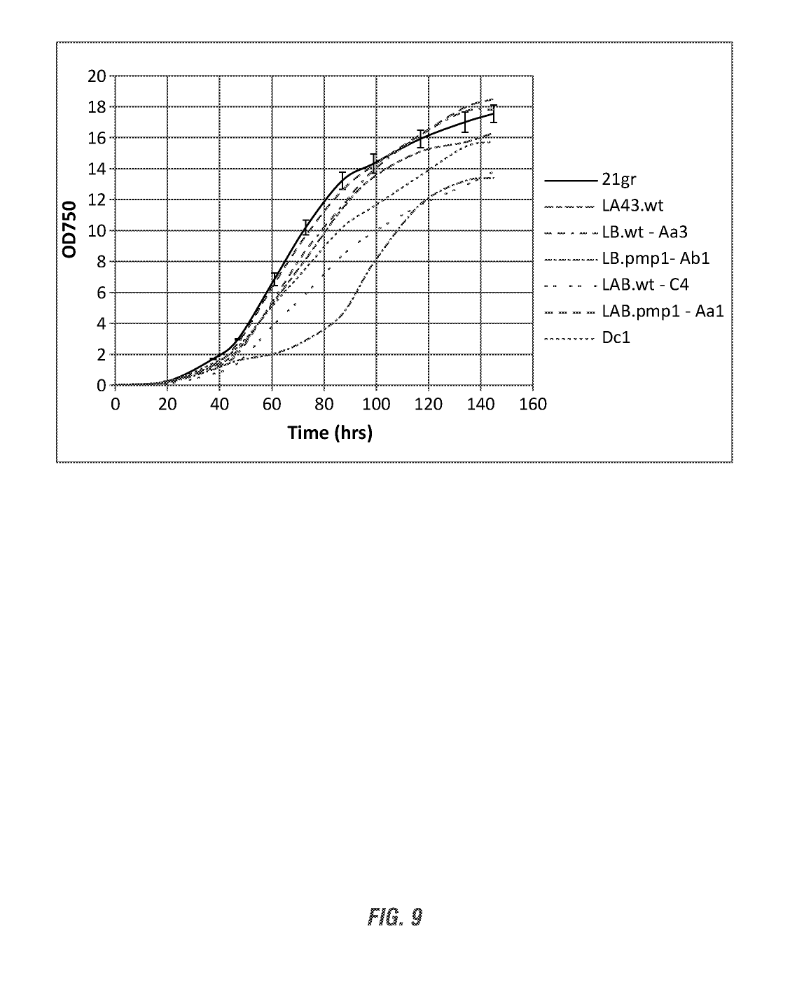

FIG. 9. Additional data from experiment illustrated in FIG. 5. Cell density estimates for wild-type and transgenic Chlamydomonas strains determined over the course of culture growth in 200 ml mini-bioreactors. Transgenic lines include the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background in two independent progeny (LAB.wt-C4 and LAB.Dc1) and the LCIA and LCIB transgenes both in an lcib mutant background (LAB.pmp1-Aa1). Samples were removed at frequent intervals, diluted into a measurable range and the OD₇₅₀ determined in a UV/Vis spectrophotometer. Values for each strain represent the average from two separate cultures in different mini-bioreactors, except that for 21gr, which represents the average of several separate cultures.

### Example 2

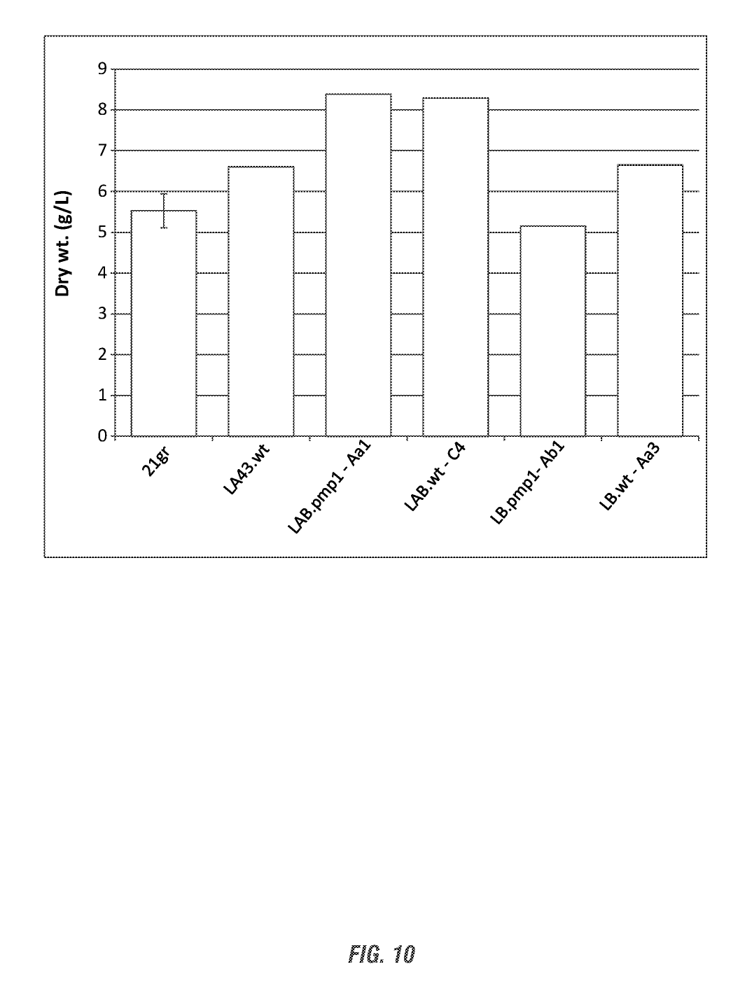

A transgenic Chlamydomonas lcib mutant line expressing an inserted LCIB gene regulated by a high expression, constitutive promoter was crossed with a transgenic Chlamydomonas line expressing an inserted LCIA gene regulated by a high expression, constitutive promoter. Progeny from this cross with different genotypes, including the LCIA transgene in a WT background (LA43.wt), the LCIB transgene in a WT background (LB.wt-Aa3), the LCIB transgene in an lcib mutant background (LB.pmp-Ab1), the LCIA and LCIB transgenes both in a WT background (LAB.wt-C4) and the LCIA and LCIB transgenes both in an lcib mutant background (LAB.pmp1-Aa1), were grown along with WT (21gr) in 200 ml photobioreactors to stationary phase, then harvested by centrifugation and the cell pellets dried for biomass determination. The results are shown in FIG. 10.

### REFERENCES

### All are Hereby Incorporated in their Entirety Herein by Reference

1. Spalding M. H. In: the Molecular Biology of Chloroplasts and Mitochondria in Chlamydomonas. Rochaix J. D., Goldschmidt-Clermont M., Merchant S., editors. Dordrecht, The Netherlands: Kluwer; 1998. pp. 529-547.
2. Kaplan A., Reinhold L. Annu. Rev. Plant Physiol. Plant. Mol. Biol. 1999; 50:539-559.
3. Badger M. R., Price G. D. J. Exp. Bot. 2003; 54:609-622.
4. Xiang Y., Zhang J., Weeks D. P. Proc. Natl. Acad. Sci. USA. 2001; 98:5341-5346.
5. Funke R. P., Kovar J. L., Weeks D. P. Plant Physiol. 1997; 114:237-244.
6. Fukuzawa H., Miura K., Ishizaki K., Kucho K., Saito T., Kohinata T., Ohyama K. Proc. Natl. Acad. Sci. USA. 2001; 98:5347-5352.
7. Karlsson J., Clarke A. K., Chen Z. Y., Hugghins S. Y., Park Y. I., Husic H. D., Moroney J. V., Samuelsson G. EMBO J. 1998; 17:1208-1216.
8. Mamedov T. G., Suzuki K., Miura K., Kucho K., Fukuzawa H. J. Biol. Chem. 2001; 276:45573-45579.
9. Nakamura Y., Kanakagiri S., Van K., Spalding M. H. Can. J. Bot. 2005; 83:796-809.
10. Pollock S. V., Colombo S. L., Prout D. L., Jr, Godfrey A. C., Moroney J. V. Plant Physiol. 2003; 133:1854-1861.
11. Moroney J. V., Husic H. D., Tolbert N. E., Kitayama M., Manuel L. J., Togasaki R. K. Plant Physiol. 1989; 89:897-903.
12. Spalding M. H., Spreitzer R. J., Ogren W. L. Plant Physiol. 1983; 73:268-272.
13. Spalding M. H., Spreitzer R. J., Ogren W. L. Plant Physiol. 1983; 73:273-276.
14. Miura K., Yamano T., Yoshioka S., Kohinata T., Inoue Y., Taniguchi F., Asamizu E., Nakamura Y., Tabata S., Yamato K. T., et al. Plant Physiol. 2004; 135:1595-1607.
15. Van K., Wang Y., Nakamura Y., Spalding M. H. Plant Physiol. 2001; 127:607-614.
16. Spalding M. H., Van K., Wang Y., Nakamura Y. Funct. Plant Biol. 2002; 29:221-230.
17. Vance P., Spalding M. H. Can. J. Bot. 2005; 83:820-833.
18. Lumbreras V., Stevens D. R., Purton S. Plant J. 1998; 14:441-448.
19. Chen Z.-Y., Lavigne M. D., Mason C. B., Moroney J. V. Plant Physiol. 1997; 114:265-273.
20. Fujiwara S., Fukuzawa H., Tachiki A., Miyachi S. Proc. Natl. Acad. Sci. USA. 1990; 87:9779-9783.
21. Geraghty A. M., Anderson J. C., Spalding M. H. Plant Physiol. 1990; 93:116-121.
22. Kindle K. L. Proc. Natl. Acad. Sci. USA. 1990; 87:1228-1232.
23. Harris E. H. The Chlamydomonas Source Book: A Comprehensive Guide to Biology and Laboratory Use. San Diego: Academic; 1989.
24. Badger M. R., Kaplan A., Berry J. A. Plant Physiol. 1980; 66:407-413.
25. Moroney J. V., Husic H. D., Tolbert N. E. Plant Physiol. 1985; 79:177-183.
26. Ausubel F. M., Brent R., Kingston R. E., Moore D. D., Seidman J. G., Smith J. A., Struhl K. Current Protocols in Molecular Biology. New York: Greene & Wiley; 1987.
27. Chomczynski P., Sacchi N. Anal. Biochem. 1987; 162:156-159.
28. Sambrook J., Fristch E. F., Maniatis T. Molecular Cloning: A Laboratory Manual. 2nd Ed. Plainview, N.Y.: Cold Spring Harbor Lab. Press; 1989.
29. Fischer N., Rochaix J. D. Mol. Genet. Genomics. 2001; 265:888-894.
30. Shrager J., Hauser C., Chang C.-W., Harris E. H., Davies J., McDermott J., Tamse R., Zhang Z., Grossman A. R. Plant Physiol. 2003; 131:401-408.

### Example 3

For overexpression of lcib in Chlamydomonas, the coding region of LCIB gene was amplified by PCR from the genomic DNA and fused with a promoter amplified from the plasmid PSI103delta carrying the hsp70 enhancer element and RbcS2 promoter (Sizova et al., 2001). The final constructs includes the hsp70 promoter region (enhancer), the RbcS2 promoter, an RbcS2 intron, LCIB gene and the 3′-untranslated region.

The LCIB overexpression construct was introduced into the lcib mutant pmp-1-16-5K (Spalding et al., 1983) by electroporation. After the transformation, the putative transformants were initially screened by their growth in low CO₂ (350-400 ppm), and then the incorporation of the overexpression cassette into the genome in the putative transformants was confirmed by PCR. The lcib protein in the overexpression lines was analyzed by immunoblotting with antibodies against LCIB.

For overexpression of LCIA, the promoter region from the plasmid PSI103delta carrying the hsp70 enhancer element and RbcS2 promoter was amplified by PCR and fused with the LCIA gene. The final construct includes the promoter region, the LCIA gene, the 3′-untranslated region of the psaD gene. An expression cassette carrying the aphVIII gene PSI103delta by PCR was also cloned from the PSI103delta and ligated into the overexpression construct as the antibiotic selective marker.

The LCIA overexpression construct was introduced into the Chlamydomonas wild type strain 21gr (CC-1690) by electroporation. The putative transformants were screened by paromomycin resistance, and then the incorporation of the overexpression cassette into the genome was confirmed by PCR. The expression of LCIA was analyzed by Northern blots.

The stacking of the LCIA and LCIB overexpression cassettes into the same strain was achieved by genetic crossing between the LCIB and LCIA overexpression lines (pmpLB16×21LA43). The tetrad progenies from individual zygotes were separated and selected, and their genotypes (LCIB and LCIA overexpression cassettes in the genome) were determined by PCR analysis and immunoblotting. Results are shown in the following figures.

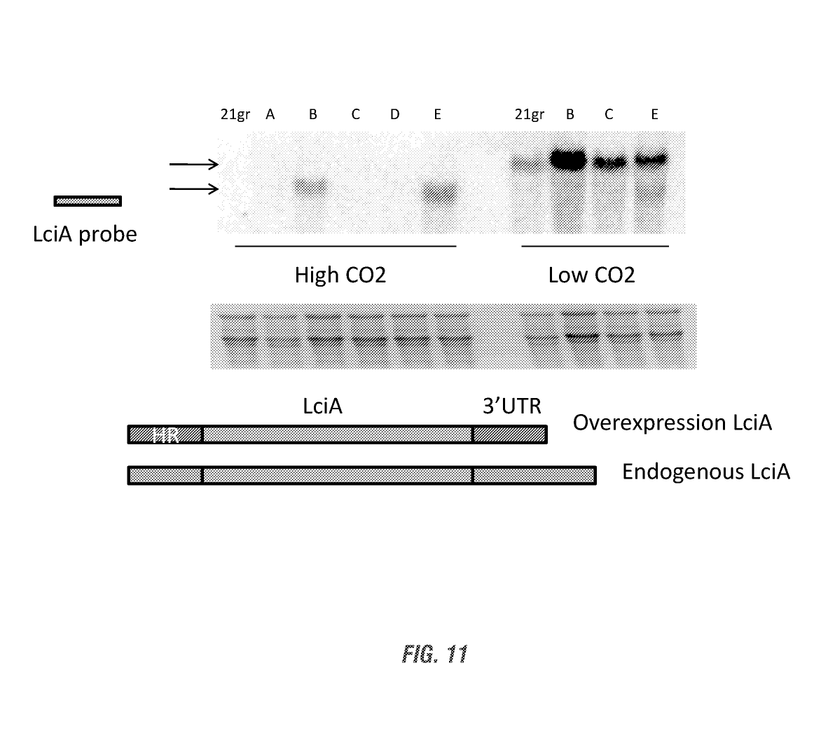

FIG. 11 shows the over-expression of LCIA in 21 gr Strain (Northern blots) as LA-A, LA-B etc. 21 gr is the wild type strain, LciA is the low CO₂ inducible gene, putative Ci transporter, and HR promoter is the Hsp70-Rbsc2 promoter.

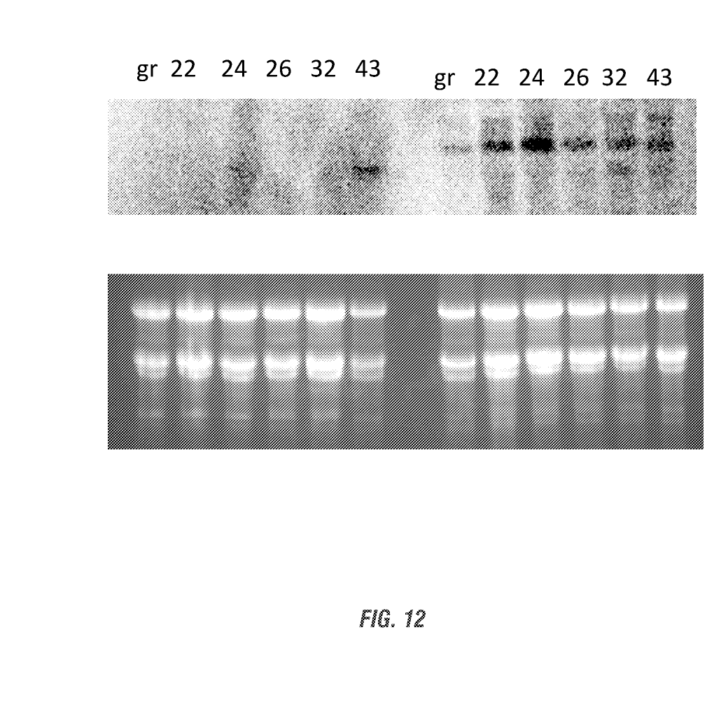

FIG. 12 shows Northern blots of LciA Gene in overexpression lines (LA22, LA23, etc.) and 21 gr (gr).

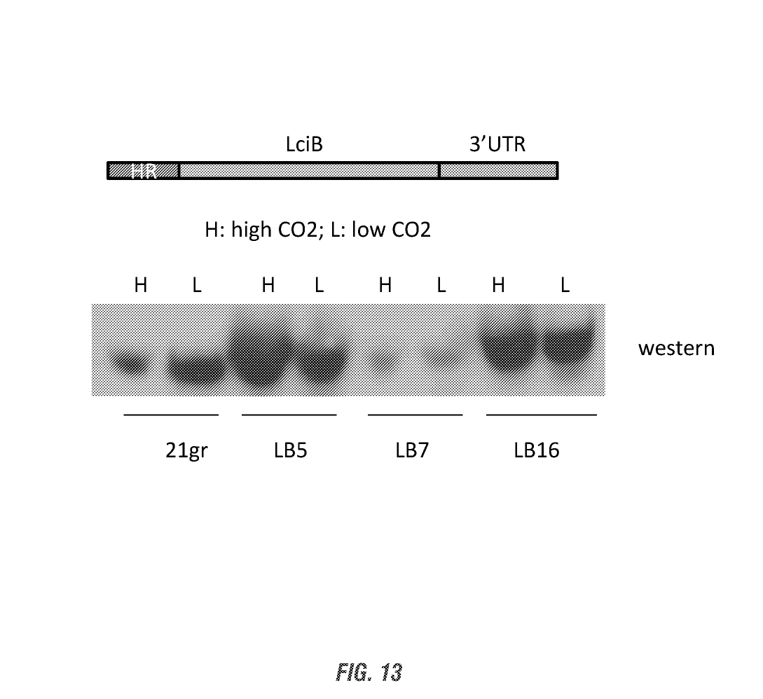

FIG. 13 shows over-expression of LciB in pmp1 strain. Lci-B=a novel protein involved in inorganic cargon (Ci) accumulation pmp1-Lcib mutant (wild type 137c background, 21gr-wild type strain, HR promoter—Hsp70-Rbcs2 promoter.

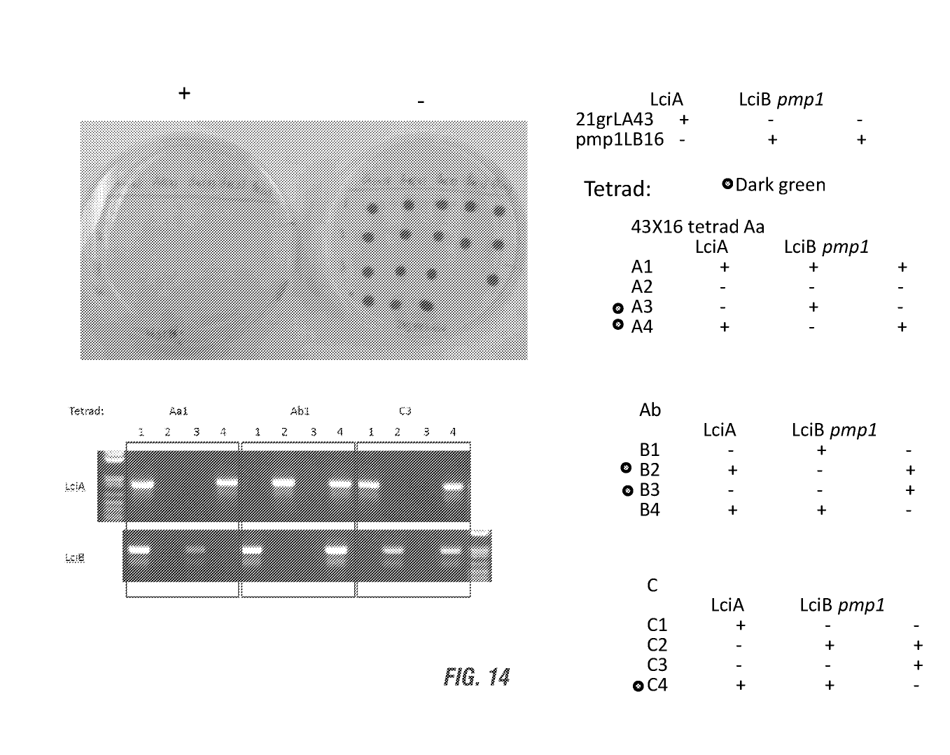

FIG. 14 shows 21grLA-43×pmp1LB-16, PCR LciB: RbcSa-LciBas; LciA: LciAa-PsaDas.

FIG. 15 shows Progeny from cross (21grLA-43×pmp1LB-16) and their LCIB expression levels in western blots.

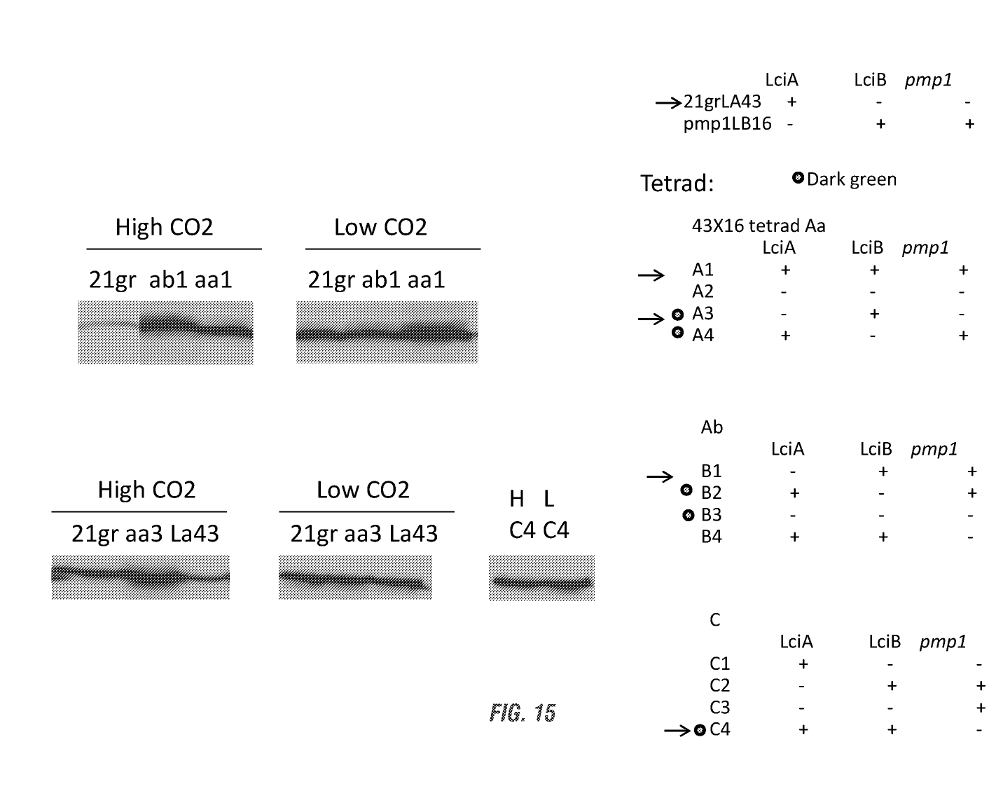

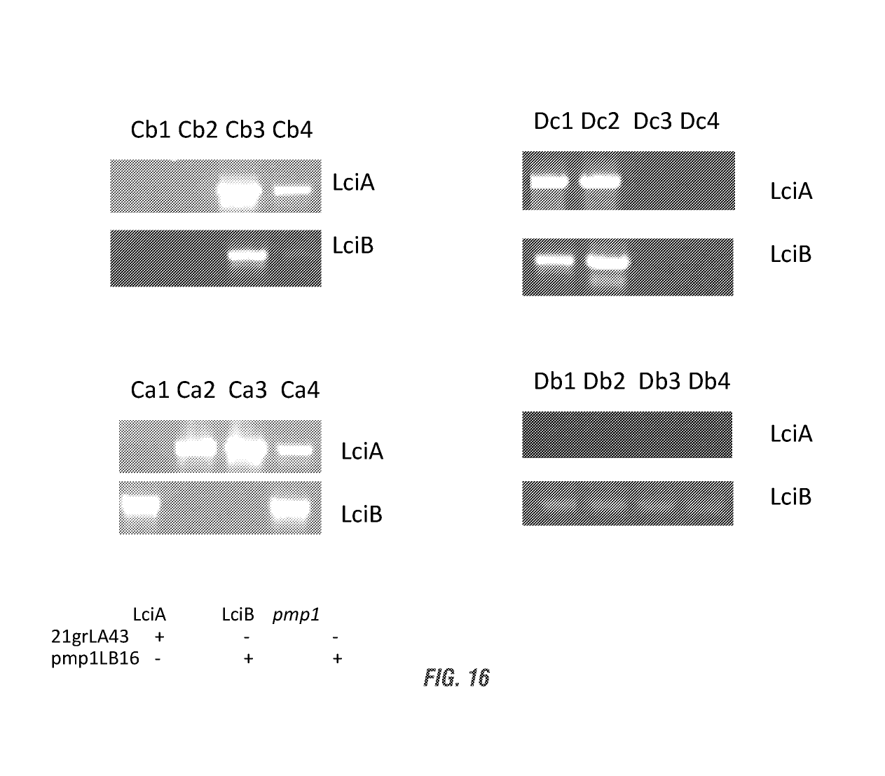

FIG. 16 shows Progeny from 21grLA-43×pmp1LB-16, PCR detection of LCIA and LCIB.

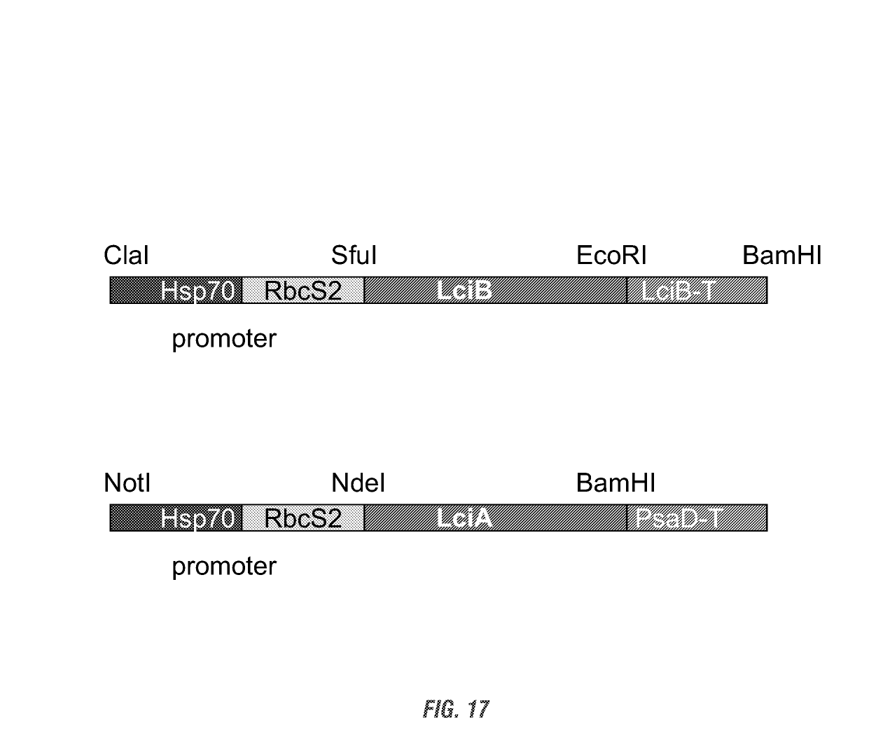

FIG. 17 shows overexpression of LCIB and LCIA, sfu/bamHi LciB into psp103, use Kpn1 to linearize.

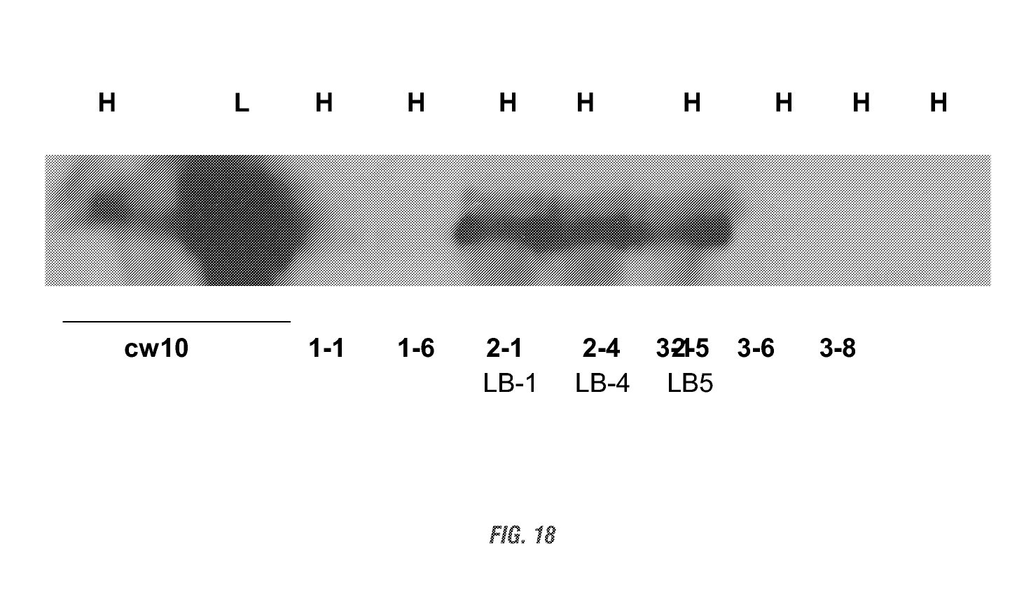

FIG. 18 shows overexpression of LCIB in wild-type cw10.

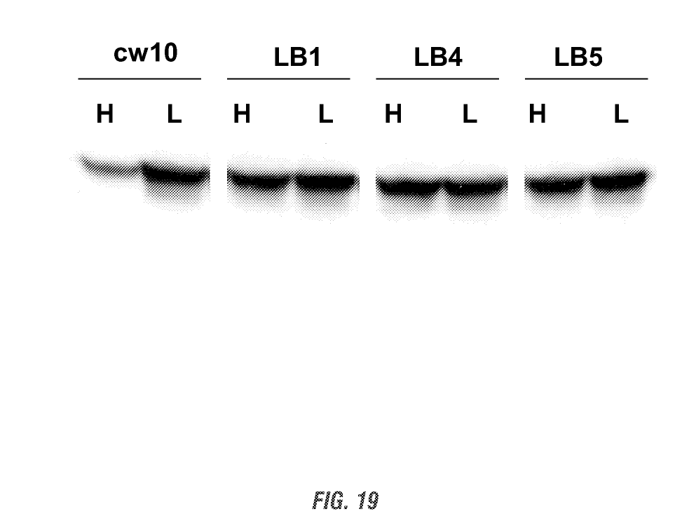

FIG. 19 shows overexpression of LCIB in cw10-western blots of LCIB.

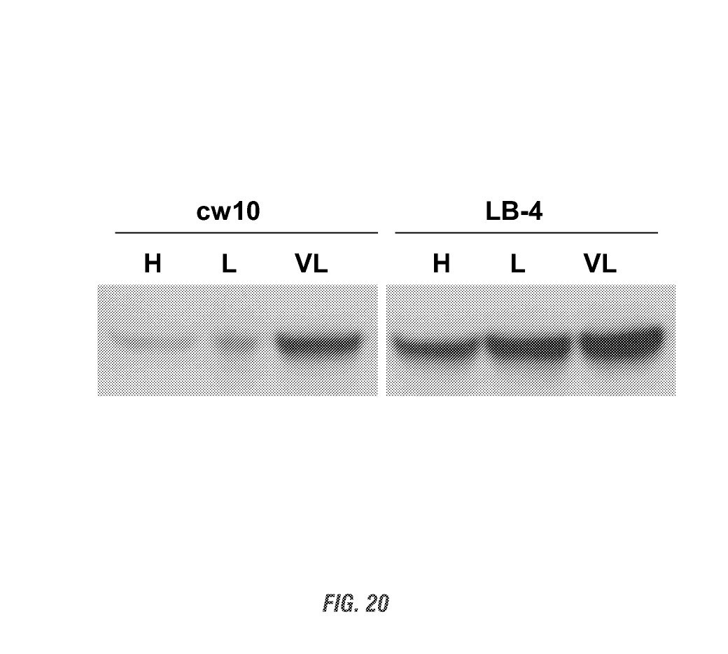

FIG. 20 shows overexpression of LCIB in cw10-western blots of LCIB.

FIG. 21 shows photosynthetic oxygen evolution.

### Engineer Increased CCM Activity

We have transgenic lines from transformations with LCIA and LCIB (and both LCIA and LCIB combined) documented as expressed, and we have transformations with LCI1, RHP1, CAH1 and HLA3 in the process of analysis to confirm expression.

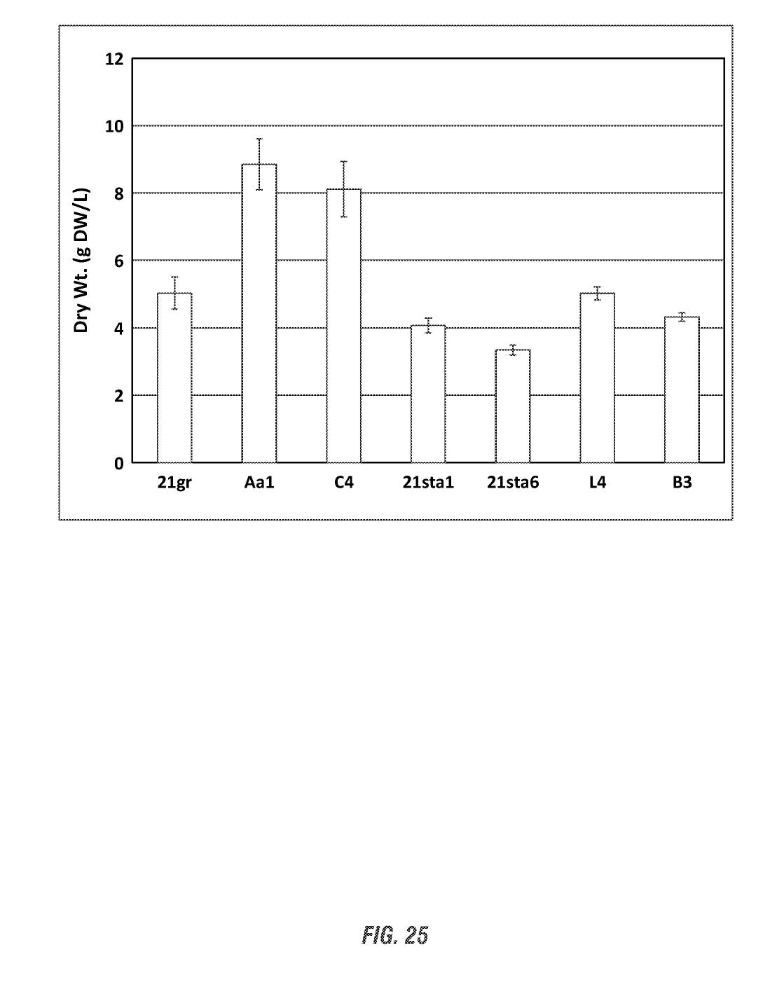

We have documented growth and biomass accumulation increases in LCIA/LCIB transgenic combinations of up to ˜80% relative to 21gr (see FIG. 24), and we have demonstrated that most of this increase in biomass can be explained as an increase in starch accumulation (see FIG. 25).

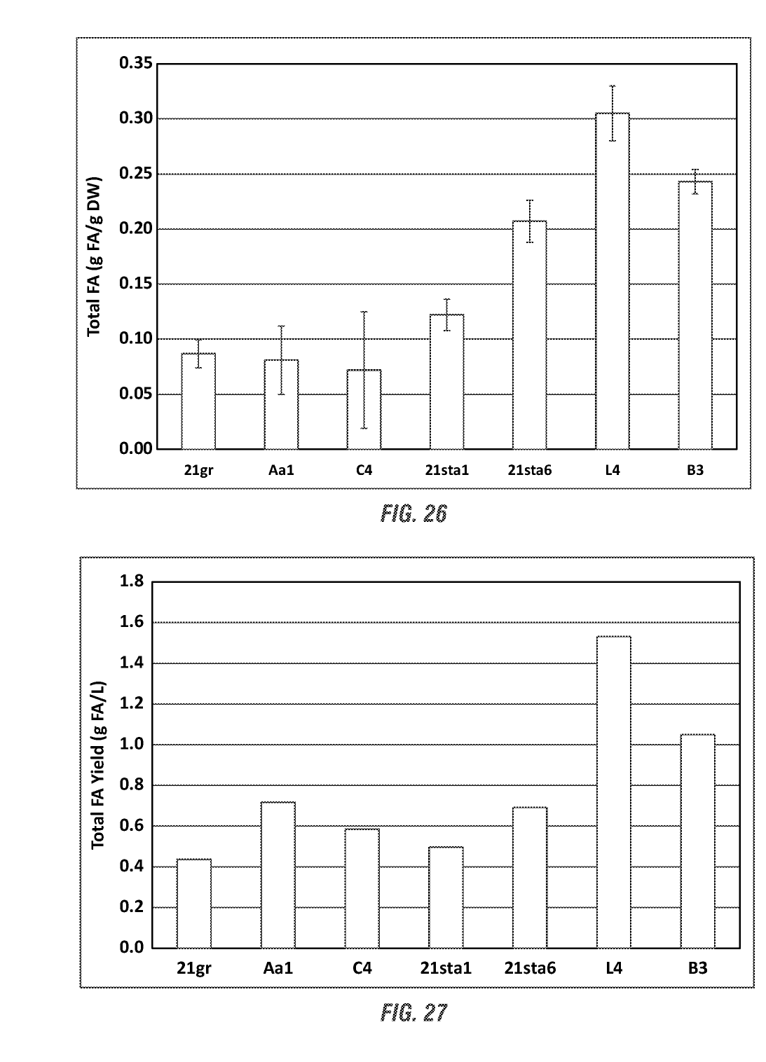

We have characterized photosynthesis in an LCIA/LCIS double transgenic (Dc2) to confirm an increase in photosynthetic performance (see FIG. 26). These experiments have demonstrated increased photosynthesis rates over a range of CO₂ concentrations in high-CO₂ acclimated cells, including a >35% higher photosynthetic rate (142±15 vs. 104±5 μmoles O₂ mg⁻¹ Chl h⁻¹; P<0.02) in in near-saturating CO₂ concentrations (1000 μM NaHCO₃, pH 7.0) for Dc1 compared to WT 21gr. These photosynthetic measurements verify increased photosynthesis in the LCIA/LCIB transgenic line and are consistent with the increased biomass accumulation in LCIA/LCIB double transgenic lines.

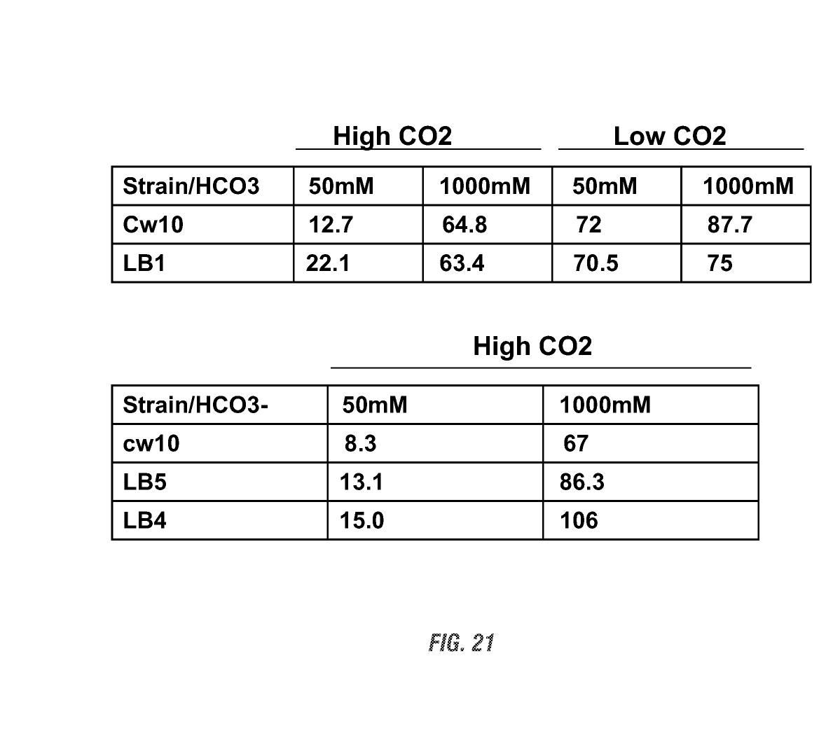

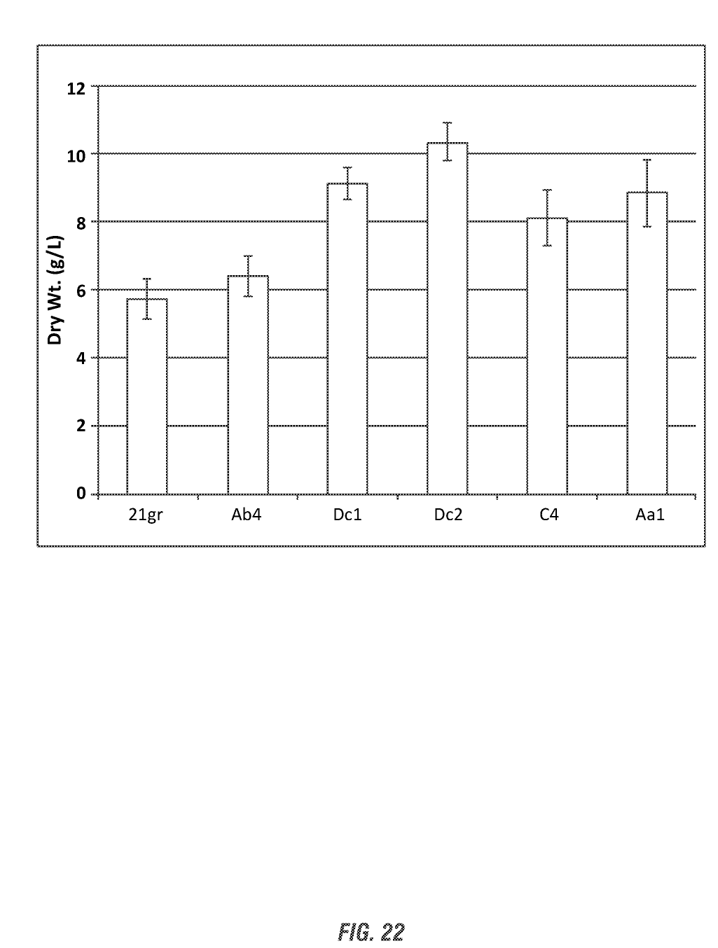

FIG. 22 shows dry biomass (g/l±std. dev.) at stationary phase for WT (21gr) compared with double transgenic lines overexpressing both LCIA and LCIB (Ab4; Dc1; Dc2; C4; Aa1). Replicated (2-4 replicates for transgenics, 12 replicates for 21gr), photoautotrophic growth in standard medium under standard conditions. All double transgenics, except Ab4, are significantly different from 21gr at P<0.01.

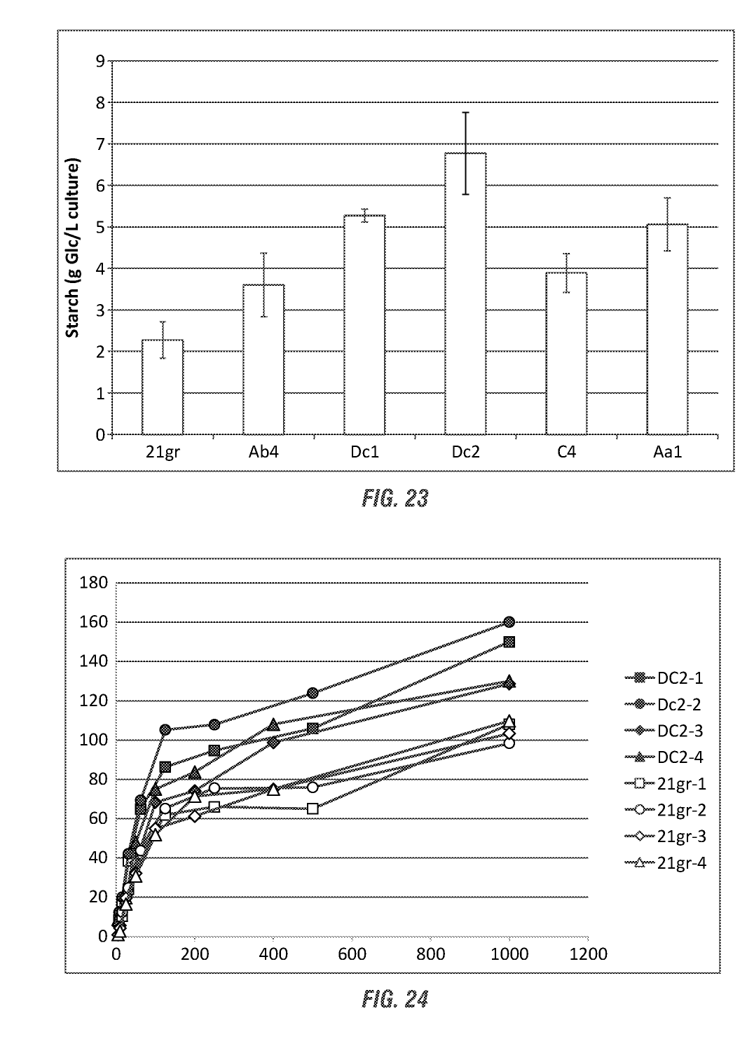

FIG. 23 shows starch accumulation (g/l culture±std. dev.) at stationary phase for WT (21gr) compared with double transgenic lines overexpressing both LCIA and LCIB (Ab4; Dc1; Dc2; C4; Aa1). Replicated (2-4 replicates for transgenics, 12 replicates for 21gr), photoautotrophic growth in standard medium under standard conditions. All double transgenics are significantly different from 21gr at P<0.01.

FIG. 24 shows Photosynthetic rate measured as CO₂-dependent O₂ evolution (μmoles O₂ mg⁻¹ Chl h⁻¹) as a function of the dissolved inorganic carbon concentration (μM NaHCO₃, pH 7.0) for high-CO₂ acclimated WT (21gr; blue) compared with double transgenic line Dc2 (red) overexpressing both LCIA and LCIB. Four replicates from two independent experiments are shown.

We have increased not only total biomass (dry weight—DW) yield (g DW/L), but we also have increased total fatty acid (FA) content (g FA/g DW; FIG. 22) and total FA yield (g FA/l culture; FIG. 23) far beyond any previous levels demonstrated for photoautotrophic Chlamydomonas, including the starch-deficient sta1 mutant and the newly incorporated starchless sta6 mutant.

By stacking (combining genetically) the starchless mutation, sta6, with two independent transgenes, overexpression of LCIA and overexpression of LCIB, we have increased total FA content to >24% (>0.24 g FA/g DW) in line B3 and to >30% (>0.30 g FA/g DW) in line L4. The FA content of L4 is 250% higher than that of 21gr (B3 is 179% higher), 150% higher than that of 21sta1 (B3 is 100% higher), and 47% higher than that of 21sta6 (B3 is 17% higher).

Please note that our “lipid” analyses depend on GC analysis of total fatty acid content, which give substantially lower values than more commonly used gravimetric measurements of total lipid content. However, measurement of total FA content provides more accurate and realistic values relative to usable hydrocarbon content. Total lipid measurements include chlorophyll and other non-fatty acid derived lipids, as well as the non-hydrocarbon portions of polar membrane lipids, such as MGDG and DGDG. The fatty acid portion represents only about 50% of the total mass of DGDG; the other half of the mass comes from sugars (galactose) and glycerol.

In addition to increased FA content, the combination of increased FA content (FIG. 22) with increased biomass has resulted in a stunning increase in total FA yield in the stacked genotypes. Double transgenic/sta6 lines L4 and B3 have FA yields of >1.5 g FA/1 and >1.0 g FA/1, which are 248% (L4) and 139% (B3) higher than 21gr, 206% (L4) and 110% (B3) higher than 21sta1, and 122% (L4) and 52% (B3) higher than 21sta6. The total FA yield of >1.5 g/l is as high as the total biomass yield of the WT strain (137c) we were using at the beginning of this project.

### Engineer Increased CCM Activity

We have overexpressing transgenic lines from transformations with LCIA and LCIB (and both LCIA and LCIB combined), and we have confirmed expression in transgenic LCI1, and CAH1. Transgenic RHP1 and HLA3 lines are in the process of analysis to confirm expression.

We have documented growth and biomass accumulation increases in LCIA/lcib transgenic combinations of up to ˜80% (average of ˜50% increase over 5 different double transgene lines) relative to 21gr and we have demonstrated that much of this increase in biomass can be explained as an increase in starch accumulation. We also have confirmed that photosynthetic rate (measured as CO₂-dependent O₂ evolution) in near-saturating CO₂ concentrations (1000 μM NaHCO₃, pH 7.0) is increased by about 35% in the LCIA/LCIB double transgenic Dc2. These photosynthetic measurements verify increased photosynthesis in the LCIA/LCIB transgenic line and are consistent with the increased biomass accumulation in LCIA/LCIB double transgenic lines.

In addition to the photosynthesis rates measured as O₂ evolution, we also are currently measuring photosynthesis in WT (21gr) and in the LCIA/LCIS double transgenic (Dc2) in situ (in photobioreactors) during growth of the cultures by measuring the amount of CO₂ removed as the gas stream passes through the culture. Although the experiment is not yet completed, Dc2 clearly is removing CO₂ at a rate at least 25% higher than that of the WT strain.

We also have leveraged the increased CO₂ assimilation of the double transgenics described here to enhance FA accumulation (rather than starch accumulation) by combining these transgenes with a starch-deficient mutation (stacking the traits) as described in the opening paragraphs of this report.

FIG. 25 shows dry biomass (g DW/l±std. dev.) at stationary phase for WT (21gr) compared with double transgenic lines overexpressing both LCIA and LCIB (Aa1; C4), starch-deficient mutants, 21 sta1 and 21 sta6, and double transgenic lines overexpressing both LCIA and LCIB combined with the sta6 mutation (L4 and B3). Replicated (2-6 replicates for transgenics, >10 replicates for 21gr, 21sta1 and 21sta6), photoautotrophic growth in standard medium under standard conditions. All transgenic and mutant lines, except L4, are significantly different from 21gr at P<0.01.

FIG. 26 shows total fatty acid (FA) content (g FA/g DW±std. dev.) at stationary phase for WT (21gr) compared with double transgenic lines overexpressing both LCIA and LCIB (Aa1; C4), starch-deficient mutants, 21sta1 and 21sta6, and double transgenic lines overexpressing both LCIA and LCIB combined with the sta6 mutation (L4 and B3). Replicated (2-6 replicates for transgenics, >10 replicates for 21gr, 21sta1 and 21sta6), photoautotrophic growth in standard medium under standard conditions. All transgenic and mutant lines, except Aa1 and C4, are significantly different from 21gr at P<0.01.

FIG. 27 shows total fatty acid (FA) yield (g FA/1 culture) at stationary phase for WT (21gr) compared with double transgenic lines overexpressing both LCIA and LCIB (Aa1; C4), starch-deficient mutants, 21sta1 and 21sta6, and double transgenic lines overexpressing both LCIA and LCIB combined with the sta6 mutation (L4 and B3). Replicated (2-6 replicates for transgenics, >10 replicates for 21gr, 21sta1 and 21 sta6), photoautotrophic growth in standard medium under standard conditions.

### Example 5

### Chlamydomonas Transgenic Lines with Increased CO₂ Assimilation, High Biomass and High Lipid Yield in the Absence of Nitrogen Starvation

Introduction

We use the genetically tractable microalga Chlamydomonas reinhardtii both as a model for developing technologies to enhance microalgal production of biomass, biofuels and renewable bioproducts, and also as a potential production strain for higher value bioproducts. The advantage of Chlamydomonas rests with the use of genetic recombination to combine or stack desirable traits identified in various wild-type, mutant and transgenic strains to generate elite strains tailored to meet specific industrial needs. As a proof of concept, we have manipulated Chlamydomonas via genetic engineering, mutant screening and genetic recombination to greatly improve photosynthetic CO₂ assimilation, biomass yield and lipid yield under likely industrial algal growth conditions (high cell density and high CO₂).

Transgenic Chlamydomonas strains constitutively expressing two genes of the low-CO₂ inducible CO₂-concentrating mechanism (CCM), LCIA and LCIB, have increased biomass yield when grown at elevated CO₂ concentrations in photobioreactors, accumulating up to 10 grams dry biomass per liter.

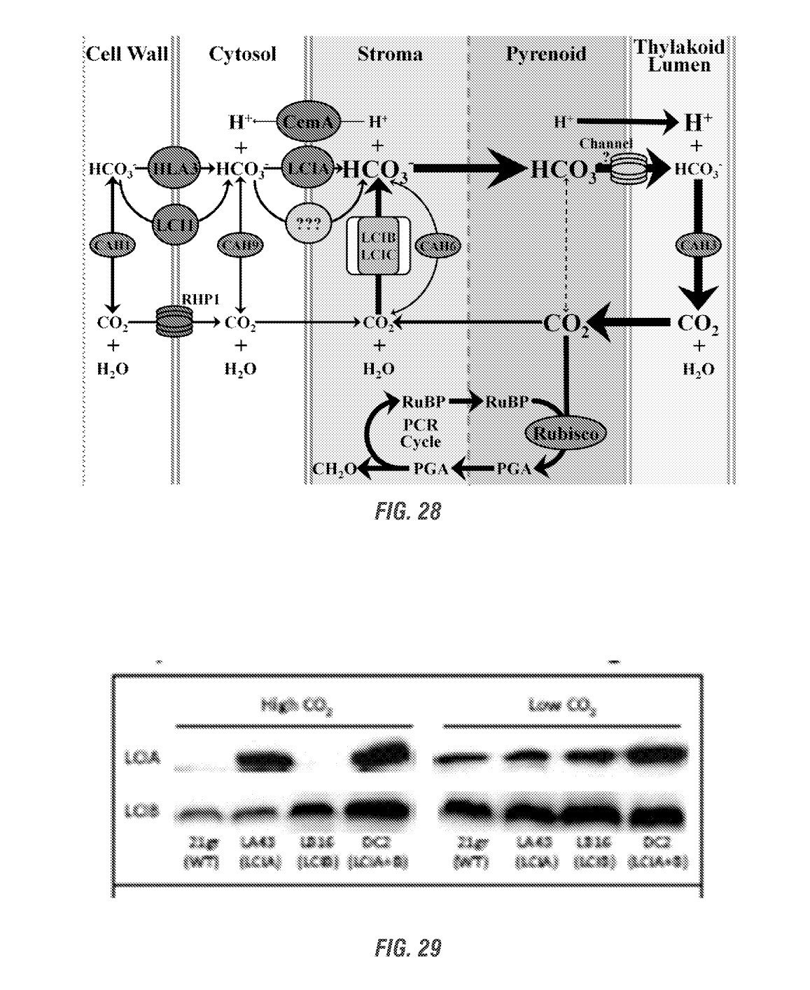

FIG. 28 is a Schematic Model of Chlamydomonas CCM. LCIA is a putative chloroplast envelope bicarbonate transporter and LCIB appears to be required for trapping CO₂ into the stromal bicarbonate pool.

FIG. 29 (Expression of LCIA and LCIB in Transgenics) is a Western blot analysis of LCIA and LCIB expression in wild type (WT) 21gr and over-expression strains LA43 (over-expressing LCIA), LB16 (over-expressing LCIB), and Dc2 (over-expressing LCIA and LCIB).

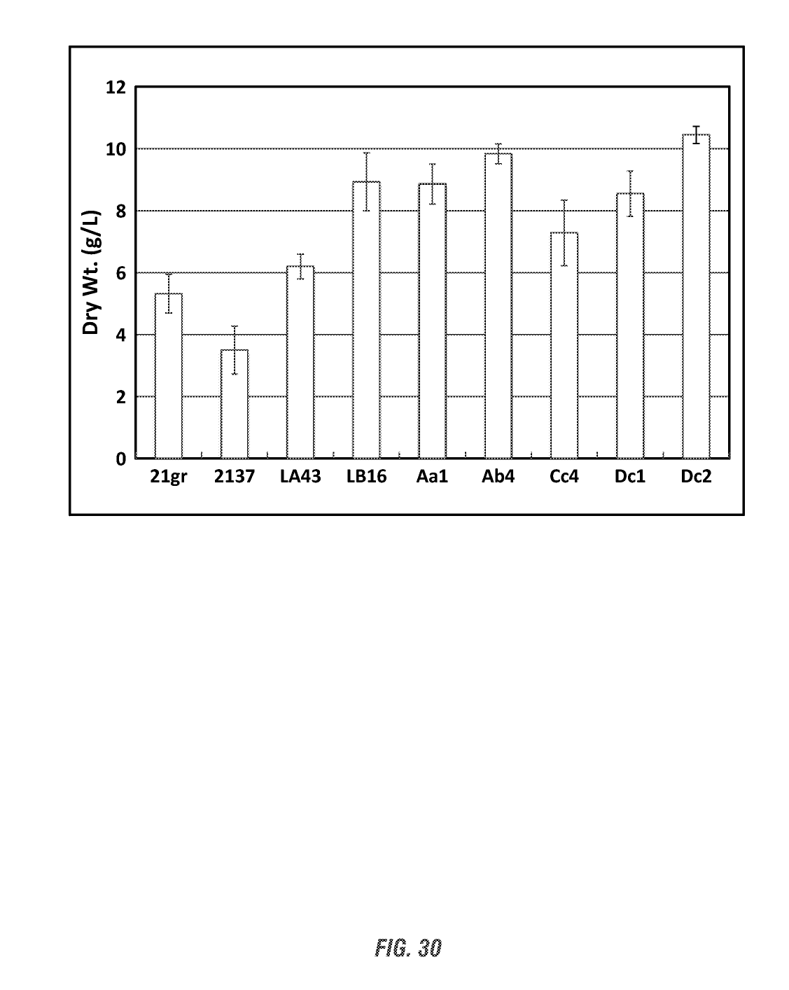

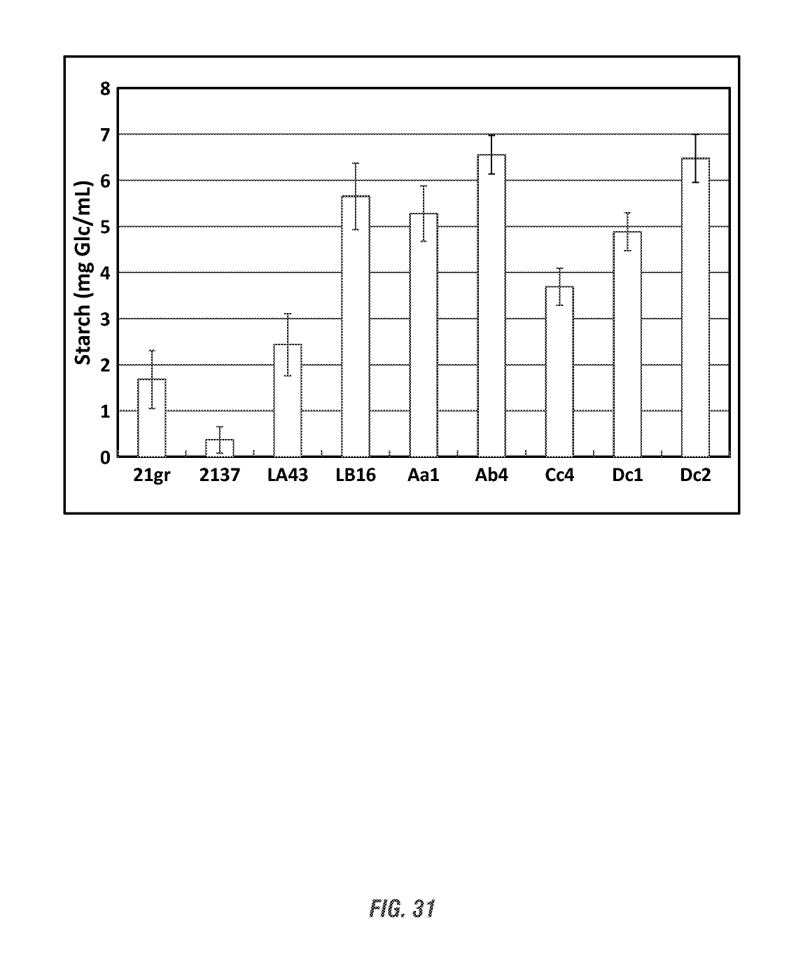

FIG. 30 (Biomass and Starch Yield in Transgenics) is a graph showing that transgenes LCIA and LCIB increase biomass yield.

FIG. 31 is a graph showing extra biomass of transgenics accumulates as starch.

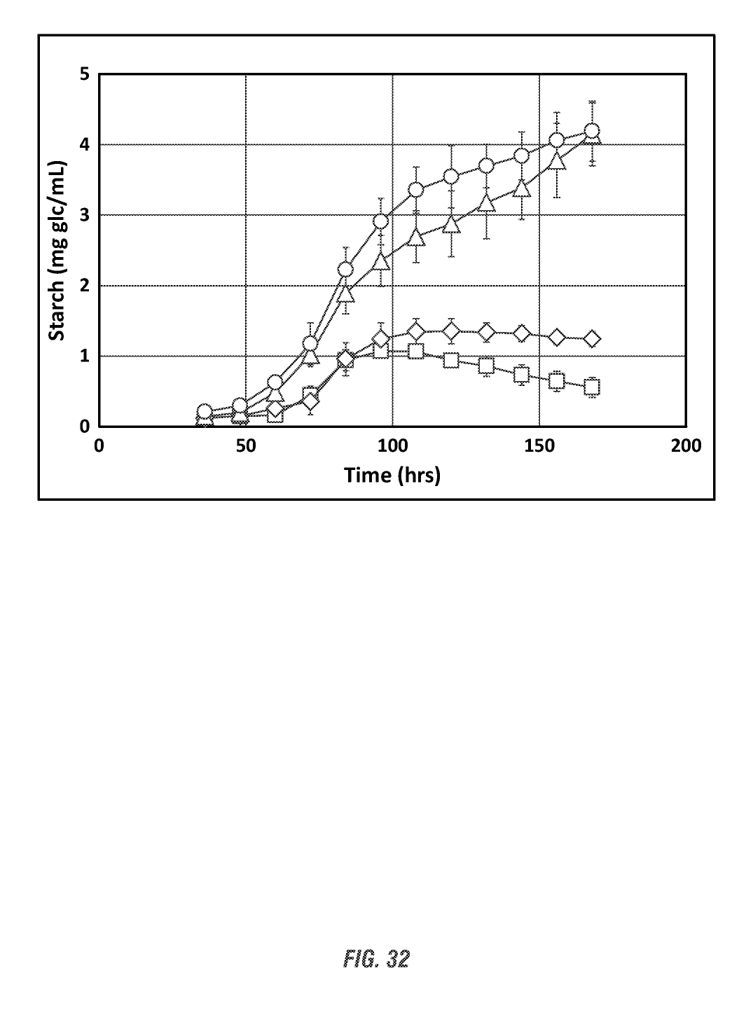

FIG. 32 is a graph showing that increased starch accumulates throughout growth of the culture, without nitrogen starvation. A. Biomass yield of photoautotrophic cultures at stationary phase for wild-type (WT) strains, 21gr and 2137, single gene transgenics, LA43 (LCIA), and LB16 (LCIB), and double transgenics (LCIA+LCIB), Aa1, Ab4, Cc4, Dc1 and Dc2. B. Starch content of photoautotrophic cultures at stationary phase for WT, 21gr and 2137, single gene transgenics, LA43 (LCIA), and LB16 (LCIB), and double transgenics (LCIA+LCIB), Aa1, Ab4, Cc4, Dc1 and Dc2. C. Starch content during photoautotrophic growth of cultures for WT 21gr and transgenics LA43 (LCIA), LB16 (LCIB) and Dc2 (LCIA+LCIB).

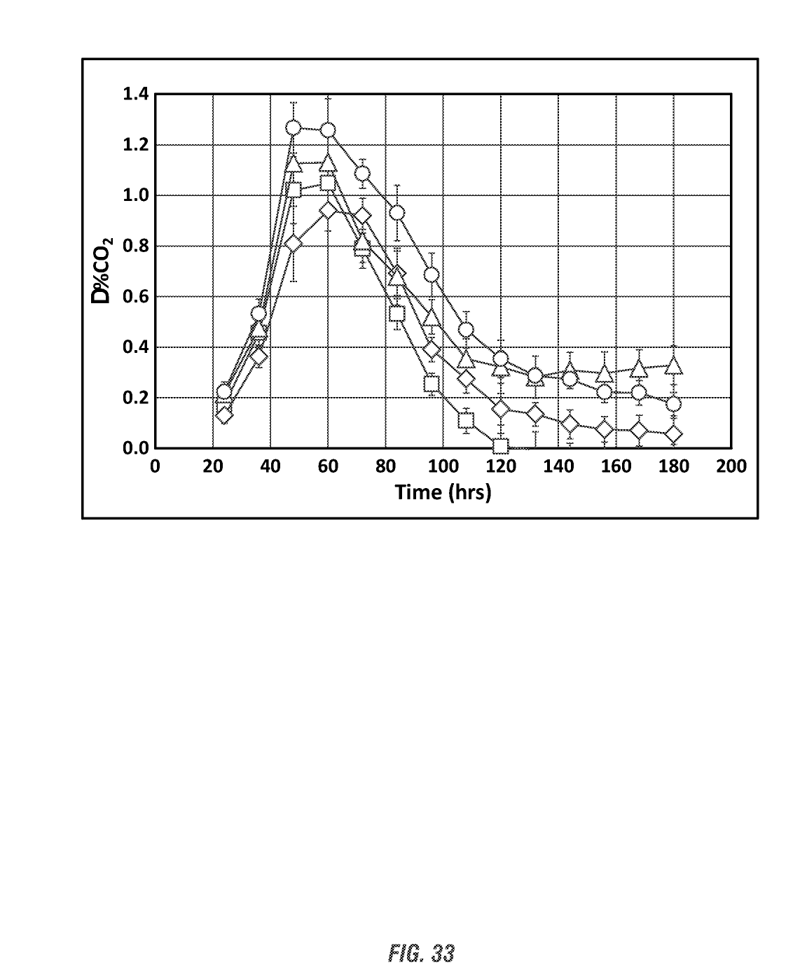

FIG. 33 is a graph showing Increased CO₂ Assimilation in Transgenics. Whole-bioreactor CO₂ assimilation increases in Transgenics.

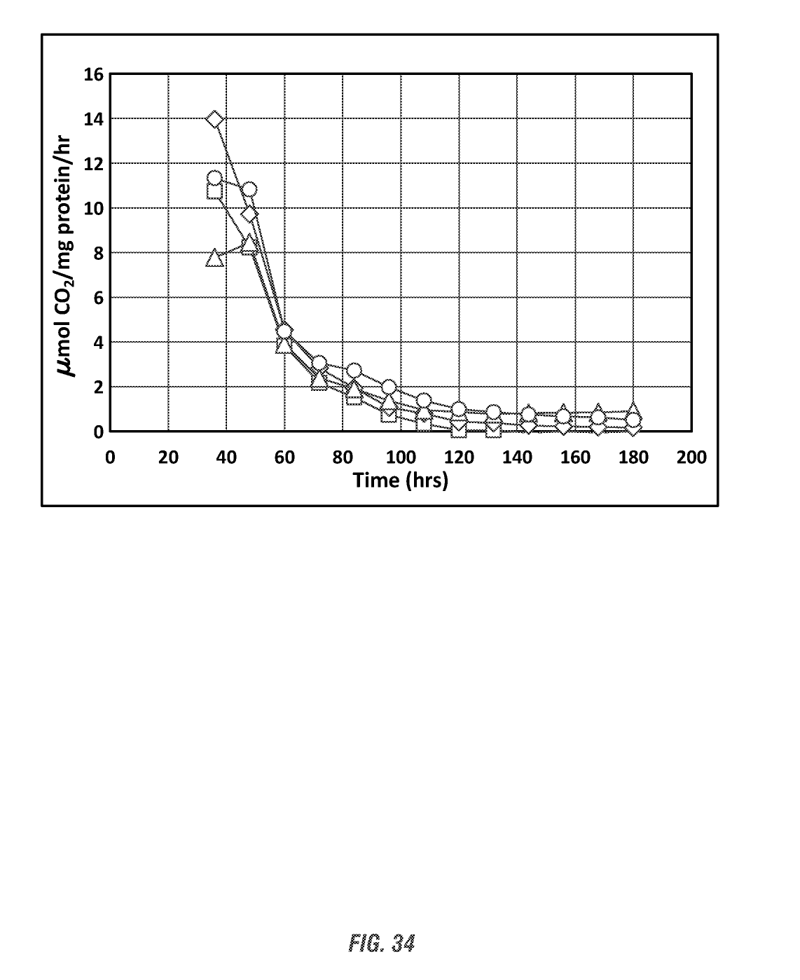

FIG. 34 is a graph showing CO₂ assimilation rate per mg protein is increased in transgenics.

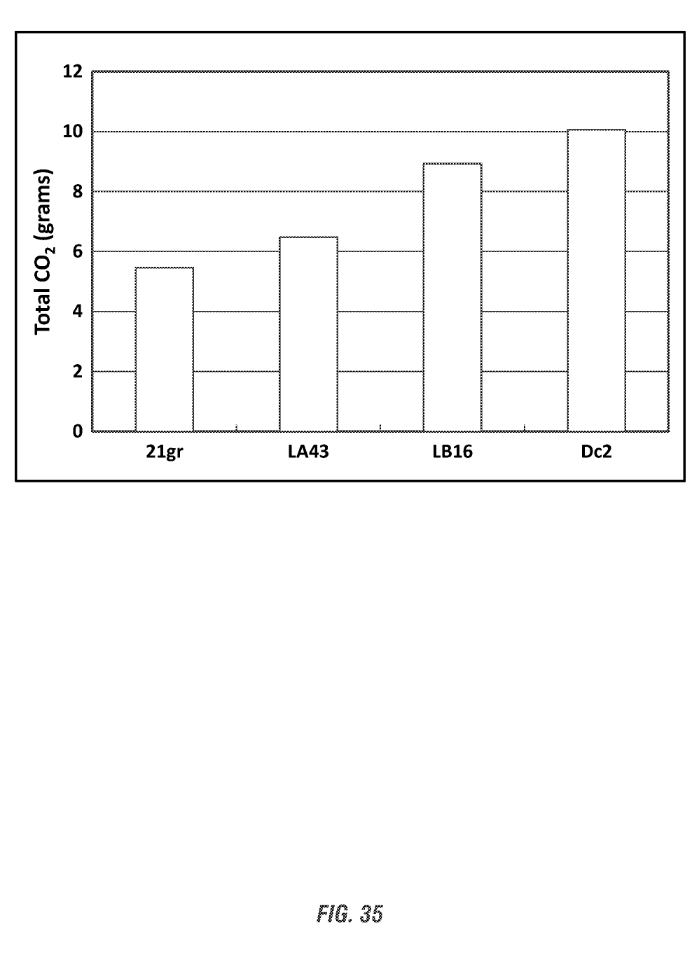

FIG. 35 is a graph showing total CO₂ assimilated is increased in transgenics. A. measurement of net, direct, in situ uptake of CO₂ by WT 21gr and transgenics LA43 (LCIA), LB16 (LCIB) and Dc2 (LCIA+LCIB) in photobioreactors. B. Calculated rate of net in situ CO₂ assimilation per mg of protein for WT 21gr and transgenic lines LA43 (LCIA), LB16 (LCIB) and Dc2 (LCIA+LCIB). C. Total in situ net CO₂ assimilation for WT 21gr and transgenics LA43 (LCIA), LB16 (LCIB) and Dc2 (LCIA+LCIB) over the full growth of cultures in photobioreactors.

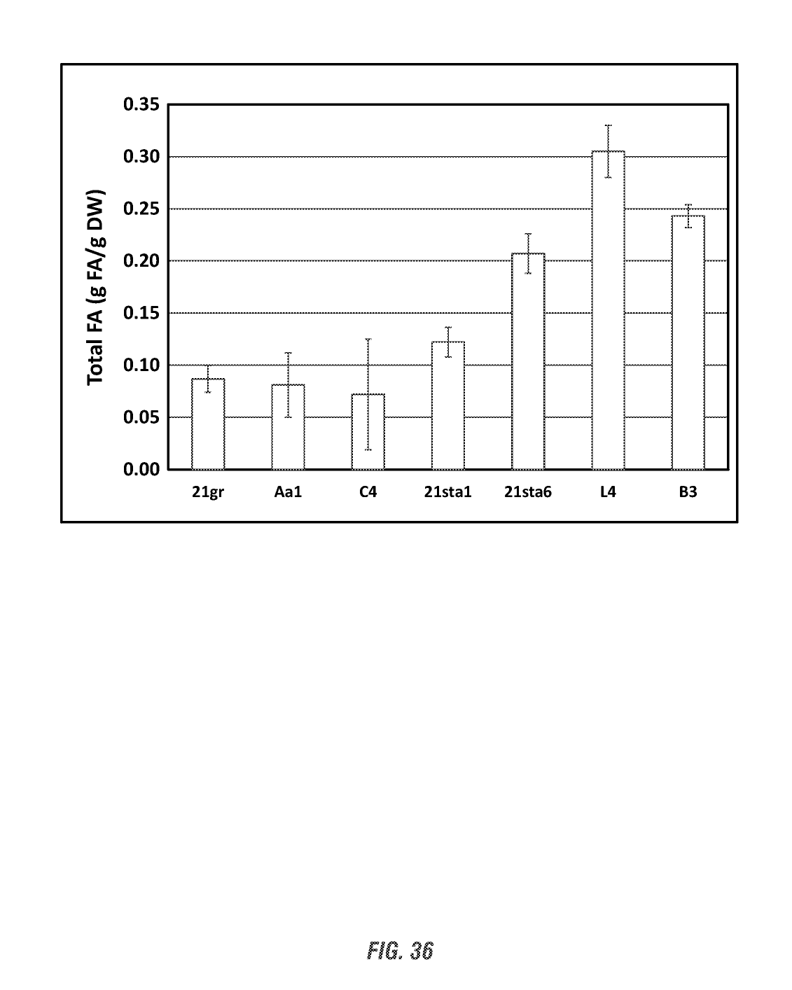

FIG. 36 is a graph showing that adding a Starch-less Mutation Channels the Extra Carbon into Fatty Acids. Fatty acid (FA) content increases in starch synthesis mutants st1 and st6, as well as in double transgenic Aa1 crossed with st6.

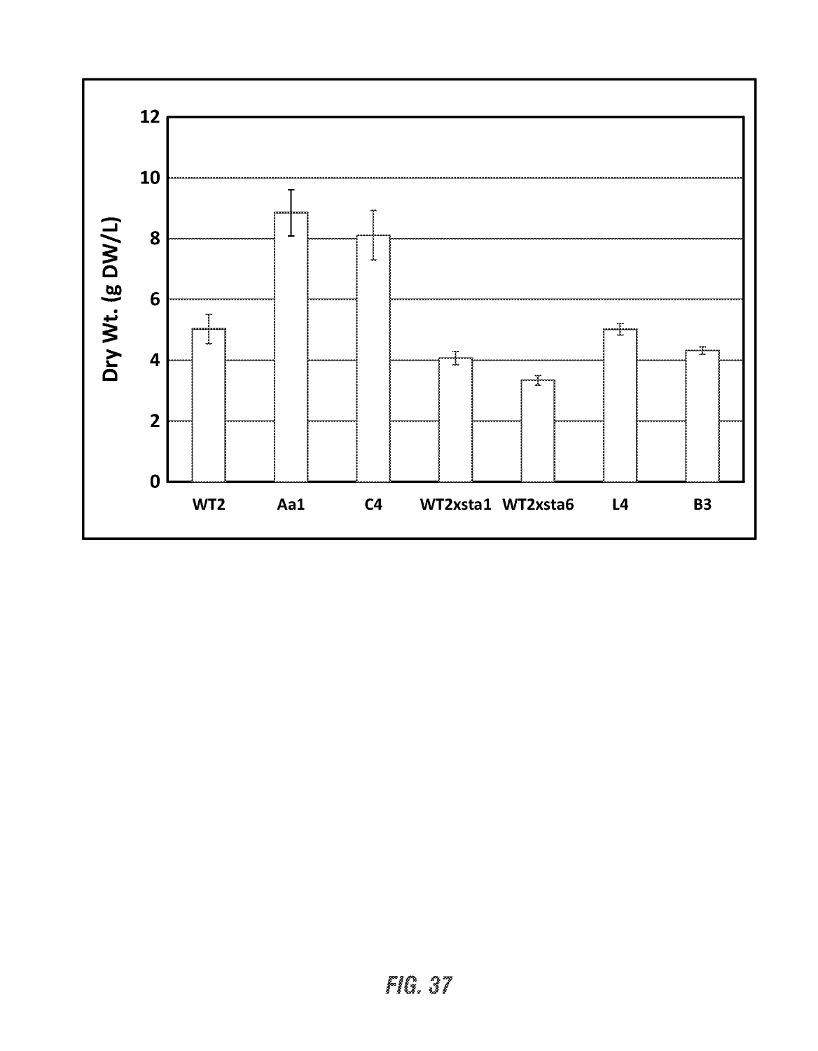

FIG. 37 is a graph showing that biomass decreases because synthesis of 1.5 g of oil requires the same CO₂ assimilation as 5 g of starch.

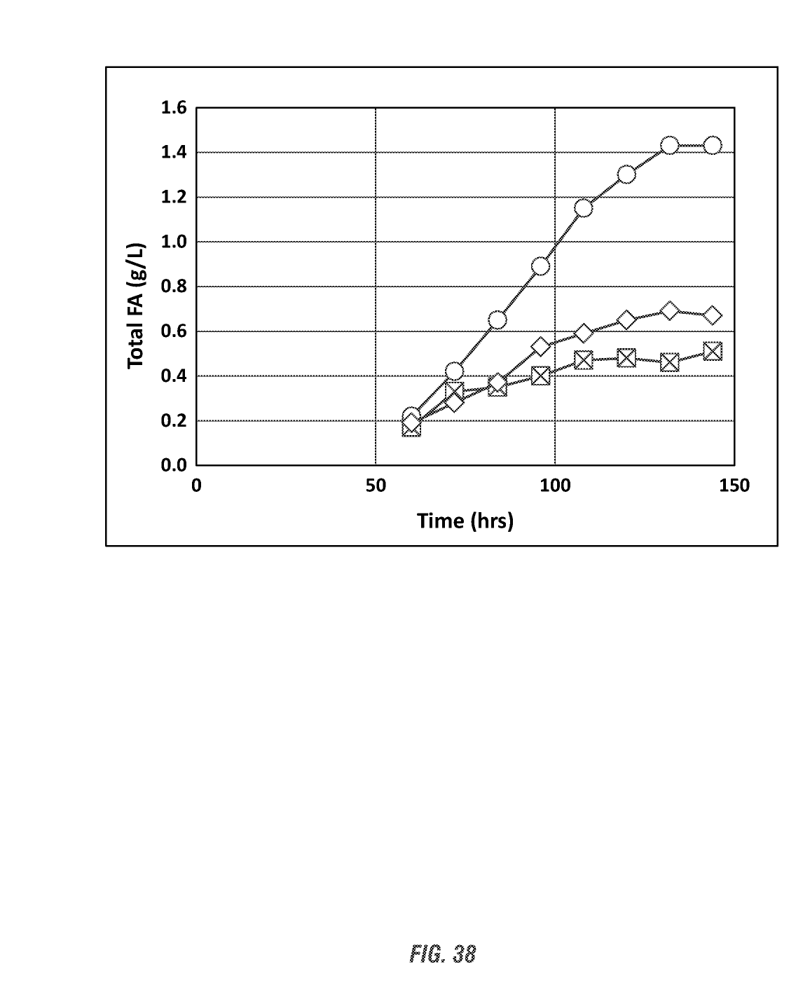

FIG. 38 is a graph showing that is Increased FA accumulates throughout growth of the culture, without nitrogen starvation.

Summary
Over-expression of transgenes LCIA or LCIB increases biomass accumulation 15-70%
Co-over-expression of both transgenes LCIA and LCIB increases biomass accumulation 40-80%
Increased biomass results from increased CO₂ assimilation and yields mainly increased starch
Genetically blocking starch synthesis in double transgenic lines converts starch accumulation to oil accumulation (increase to 30% total FA from only 8% total FA in WT 21gr)
Oil yield increases from ˜0.4 g FA per liter in WT 21gr to ˜1.5 g FA per liter in double transgenics
A significant advantage of these transgenic Chlamydomonas strains is that they accumulate either starch or lipid during active growth, rather than needing nutrient stress to stimulate lipid or starch synthesis.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention as described in the appended claims.

### Materials and Methods (for Figures Indicated Above):

Strains and Culture Conditions.

Chlamydomonas reinhardtii wild type strain 21gr (CC-1690) was obtained from the Chlamydomonas resource center (http://chlamycollection.org/), and the high CO₂-requiring pmp1 mutant (16-5K) has been described previously. Wild-type cells and mutant cells were maintained on agar plates with Tris-acetate-phosphate (TAP) medium (Harris 1989) and kept in Plexiglas chambers at room temperature. Liquid cultures were grown in Erlenmeyer flasks on an orbital shaker at 125 rpm. For complementation experiments requiring low CO₂ gas conditions, cells were grown on agar plates with CO₂ minimal medium (Geraghty 1990) and the gas conditions have been described previously (Wang 2006).

DNA Constructs for LCIA and LCIB Overexpression.

(a) To generate the Hsp70/RbcS promoter-LciA chimeric gene for LciA overexpression, the DNA fragment containing the Hsp70/RbcS2 hybrid promoter from the pSI103-delta plasmid (Sizova et al. 2001) was amplified by PCR with the T7 universal primer (5′-TAATACGACTCACTATAGGG-3′) and a specific primer (5′-AGGTTTCCCATATGCATTTTAAGATGTTGAGTG-3′) that introduced an NdeI restriction site at the 3′ end of the promoter. 2 kb fragment of genomic DNA containing the LciA gene was amplified by PCR with a pair of specific primers that introduced an NdeI site overhanging the start codon ATG at the 5′ end and an EcoRI site after the stop codon at the 3′ end. The NotI/NdeI-digested PCR fragment of the Hsp70/Rbcs2 promoter and the NdeI/EcoRI-digested LciA PCR fragment were ligated into the NotI/EcoRI sites in pGenD plasmid (Fischer et al. 2001). The resulted a LciA overexpression cassette including 5′-Hsp70/RbcS constitutive promoter:LciA gene:PsaD terminator-3′. The HindIII/KpnI fragment containing the AphIIIV selection marker from pSI103-delta was then inserted downstream of PsaD terminator at the HindIII/KpnI sites to complete the final pLA4 plasmid.

(b) Overexpression of LciB gene; A 2.2-kb fragment of genomic DNA containing the LciB coding region was PCR-amplified from genomic DNA with a pair of specific primers that also introduced an SfuI site before the start codon ATG at the 5′ end, and an EcoRI site after the stop codon at the 3′ end, then sub-cloned into the SfuI and BamHI sites in the plasmid pSI103-delta (Sizova et al. 2001). In the resulted plasmid LciB-sfu2, an extra sequence derived from the pSI103-delta was retained between the Hsp70/RbcS2 promoter and the cloned LciB gene. This sequence is composed of an extra start codon ATG, the first intron of the Chlamydomonas RbcS2 gene and a small piece of DNA encoding extra 13 amino acids that are originally present in pSI103-delta. It appears that these extra amino acids upstream LciB does not interfere with expression and the function of LciB, and can be cleaved with the transit peptide from the mature LCIB protein, as confirmed by the correct intracellular localization of LCIB protein in the transgenic lines.

Generation of LciA and LciB Overexpression Lines.

21gr and pmp1 were transformed with plasmid pLA4 and pLciBsfu2 respectively by electroporation. Walled cells were directly used for electroporation without autolysin treatment to remove the cell wall. Electroporation was performed as described by Shimogawara et al. (1998) with a few modifications. Briefly, cells grown mixotrophically in liquid TAP medium were harvested at early log phase (0.5−2×10⁶ cells/ml), and then resuspended in a small volume of TAP medium supplemented with 60 mM sucrose to get a final cell density of 2-4×10⁸ cells/ml. Suspended cells were placed on ice, and then 250 ul aliquots were added into each cold 4 mm electroporation cuvettes (Fisher Scientific). Before the electroporation the cuvettes were placed in 16° C. water bath for 10 minutes, and the linearized plasmid DNA was added into the cells and mixed well. Electroporation was performed with a Gene Pulser Xcell electroporator (BioRad; the condition set as 650V, 25 uF, zero resistance with exponential pulse). After the pulse, the cuvettes were kept at room temperature for at least 10 minutes. The 21gr cells transformed with pLA4 were resuspended in 15 ml TAP medium, and recovered for 24 hour with shaking before plating on TAP plates supplemented with 15 ug/ml paromomycin for selection. The pmp1 cells transformed with pLciBsfu2 were directly plated on CO₂ minimal medium agar plates without recovery and kept in air for screening the complemented cells. Although the efficiency of transformation with walled cells is far less than that with wall-less cells, we still could get reasonable amounts of transformants (up to a few hundreds per transformation) which is enough for this application.

Gene expression analysis. Colonies grown under selective conditions were transferred to fresh TAP plates for further screening. First, the presence of pLA4 or pLciBsfu2 integrated into the genome in the putative transformants was detected by colony PCR with a specific primer complementary to the Hsp70/RbcS2 promoter sequence and a primer complementary to the LciA or LciB coding sequence. For LciA overexpression, the RNA extracted from the PCR-positive transgenic lines was then analyzed by Northern blots with a gene-specific LciA probe. When grown in high CO₂, the transgenic cells reproducibly expressed LciA transgene, whereas wild type cells showed no LciA expression. When grown in low CO₂, although both transgenic lines and wild type cells expressed LciA, the expression of transgenic LciA can be distinguished from the endogenous LciA by the mRNA size change caused by different lengths of 3′UTR. Finally, the overexpression of LCIA or LCIB was analyzed by Western blots to monitor increased protein level in transgenic lines with the specific LCIA or LCIB antibodies.

Create LciA/LciB double overexpression lines by genetic cross and tetrad analysis. The genetic cross between LciA and LciB single-transgenic lines were carried out to create the LciA/LciB double overexpression lines. The mating and tetrad analysis were performed according Harris (1989). The genotype and gene expression of tetrad progeny were analyzed as with single transgenic lines.

### REFERENCES

Fischer N., Rochaix J. D. The flanking regions of PsaD drive efficient gene expression in the nucleus of the green alga Chlamydomonas reinhardtii. Mol. Genet. Genomics. 2001; 265:888-894.
Geraghty A M, Anderson J C, Spalding M H (1990) A 36-kilodalton limiting-CO₂ induced polypeptide of Chlamydomonas is distinct from the 37-kilodalton periplasmic carbonic anhydrase. Plant Physiol 93:116-121
Harris E H. (1989) The Chlamydomonas Source Book: A Comprehensive Guide to Biology and Laboratory Use (Academic, San Diego).
Spalding M H, Spreitzer R J, Ogren W L (1983b) Reduced inorganic carbon transport in a CO₂-requiring mutant of Chlamydomonas reinhardtii. Plant Physiol 73: 273-276
Sizova I, Fuhrmann M, Hegemann P (2001) A Streptomyces rimosus aphVIII gene encoding for a new type phosphotransferase provides stable antibiotic resistance to Chlamydomonas reinhardtii. Gene 277: 221-229

What is claimed is:

1. A genetically modified plant/algae/cyanobacteria having increased plant/algae/cyanobacteria photosynthesis, biomass production, and productivity in an elevated CO₂ environment, comprising a heterologous polynucleotide sequence which encodes an LCI polypeptide operably linked to a promoter sequence, wherein said modified plant/algae/cyanobacteria has increased plant/algae/cyanobacteria photosynthesis, biomass production, and productivity when compared to a non-modified plant/algae/cyanobacteria in said elevated CO₂ environment.

2. A plant part or tissue of the plant/algae/cyanobacteria of claim 1.

3. A method for increasing plant/algae/cyanobacteria photosynthesis, biomass production, and productivity in an elevated CO₂ environment comprising:
modulating the activity of a LCI protein in a plant/algae/cyanobacteria.

4. The method of claim 3 wherein said LCI protein is selected from the group consisting of one or more of: LCIA and LCIB.

5. The method of claim 1 wherein said LCI protein activity is increased.

6. The method of claim 5 wherein said LCI protein activity is increased by increasing the expression of an LCIA and/or LCIB nucleic acid.

7. The method of claim 3 further comprising the step of:
introducing into the plant/algae/cyanobacteria an expression cassette comprising a LCIA and/or LCI nucleic acid operably linked to a promoter that functions in plants/algae/cyanobacteria.

8. The method of claim 7, wherein the expression cassette is introduced by a method selected from one of the following: electroporation, micro-projectile bombardment and Agrobacterium-mediated transfer.

9. The method of claim 7 wherein the promoter that functions in plants/algae/cyanobacteria is a constitutive promoter.

10. The method of claim 3 wherein the plant/algae/cyanobacteria is Chlamydomonas reinhardtii.

11. A genetically modified plant/algae/cyanobacteria comprising a heterologous polynucleotide sequence which encodes a LCIA and/or LCIB polypeptide operably linked to a promoter sequence, wherein said modified plant/algae/cyanobacteria has increased starch production under elevated carbon dioxide conditions when compared to a non-modified plant/algae/cyanobacteria.

12. A plant part or tissue of the plant/algae/cyanobacteria of claim 11.

13. The method of claim 11 wherein said LCI protein is selected from the group consisting of one or more of: LCIA and/or LCIB.

14. The method of claim 11 wherein said LCI activity is increased in the presence of high carbon dioxide conditions.

15. The method of claim 11 wherein said LCI activity is increased by increasing the expression of an LCIA and/or LCIB nucleic acid.

16. The method of claim 11 further comprising the step of:
introducing into the plant/algae/cyanobacteria an expression cassette comprising a LCIA and/or LCIB nucleic acid operably linked to a promoter that functions in plants/algae/cyanobacteria.

17. The method of claim 16, wherein the expression cassette is introduced by a method selected from one of the following: electroporation, micro-projectile bombardment and Agrobacterium-mediated transfer.

18. The method of claim 16 wherein the promoter that functions in plants/algae/cyanobacteria is a tissue-preferred promoter, tissue-specific promoter or an inducible promoter.

19. A method for increasing plant/algae/cyanobacteria photosynthesis, biomass production, and productivity in an elevated CO₂ environment comprising:
modulating the activity of a LCI protein in a plant/algae, wherein the activity of said LCI protein is increased in an environment, or in the presence of stimulus where the LCI activity is repressed in a non-modulated plant.

20. The method of claim 19 wherein said environment is an elevated high carbon dioxide environment.
